(19) United States
(12) Patent Application Publication
(10) Pub. No.: US 2025/0283848 A1
(43) Pub. Date: Sep. 11, 2025

(54) GAS SENSOR, METHOD FOR DETERMINING ADJUSTMENT VOLTAGE TARGET VALUE FOR CONTROL OF SENSOR ELEMENT, METHOD FOR MANUFACTURING SENSOR ELEMENT, AND METHOD FOR MANUFACTURING GAS SENSOR

(71) Applicant: NGK INSULATORS, LTD., Nagoya-shi (JP)

(72) Inventors: Daichi ICHIKAWA, Nagoya-city (JP); Yusuke WATANABE, Nagoya-city (JP)

(21) Appl. No.: 19/219,316

(22) Filed: May 27, 2025

Related U.S. Application Data
(63) Continuation of application No. PCT/JP2023/031485, filed on Aug. 30, 2023.

(30) Foreign Application Priority Data
Nov. 28, 2022 (JP) 2022-188981

(51) Int. Cl. G01N 27/41; G01N 33/00

(52) U.S. Cl. CPC G01N 27/41; G01N 33/0037

(57) ABSTRACT

A gas sensor includes a sensor element and a control device, the sensor element including: a measurement pump cell, an adjustment pump cell including an inner adjustment electrode disposed in an oxygen concentration adjustment chamber, and a reference electrode. The control device is configured to control the adjustment pump cell so that an adjustment voltage that is a voltage across the reference electrode and the inner adjustment electrode reaches an adjustment voltage target value, and let V1* [mV] be the adjustment voltage target value, then following Expression (1) and Expression (2) are satisfied: V1*≤−25.7*R+436.0 (1) V1*≥−35.1*R+406.6 (2), where R=Lb*Lb/La, La is a limiting current value [mA] of an adjustment pump current which flows through the adjustment pump cell and Lb is a limiting current value [μA] of a measurement pump current which flows through the measurement pump cell.

### CROSS-REFERENCE TO RELATED APPLICATION

This application is a continuation application of PCT/JP2023/031485, filed on Aug. 30, 2023, which claims the benefit of priority of Japanese Patent Application No. JP2022-188981, filed on Nov. 28, 2022, the entire contents of which are incorporated herein by reference.

### BACKGROUND OF THE INVENTION

### 1. Field of the Invention

The present invention relates a gas sensor, a method for determining an adjustment voltage target value for control of a sensor element, a method for manufacturing a sensor element, and a method for manufacturing a gas sensor.

### 2. Description of the Related Art

Hitherto, a gas sensor that detects the concentration of a specific gas, such as NOx, in a measurement-object gas, such as the exhaust gas of an automobile, is known. For example, PTL 1 describes a gas sensor comprising: an element body which includes an oxygen-ion-conductive solid electrolyte layer, and is internally provided with a measurement-object gas flow portion that introduces a measurement-object gas and causes the measurement-object gas to flow therethrough; and a plurality of electrodes disposed in the element body. When the concentration of NOx is detected by the gas sensor, pumping-out or pumping-in of oxygen is first performed between an oxygen concentration adjustment chamber of the measurement-object gas flow portion, and the outside of a sensor element, and the oxygen concentration in the oxygen concentration adjustment chamber is adjusted. The measurement-object gas with the oxygen concentration adjusted reaches a measurement chamber provided downstream of the oxygen concentration adjustment chamber of the measurement-object gas flow portion. In the measurement chamber, the NOx in measurement-object gas is reduced in the periphery of a measurement electrode disposed in the measurement chamber. Then, feedback control is performed on a measurement pump cell so that a voltage V2 generated across the measurement electrode and a reference electrode reaches a predetermined target value, and the oxygen around the measurement electrode is pumped out. The concentration of NOx in the measurement-object gas is detected based on pump current Ip2 which flows then.

### CITATION LIST

### Patent Literature

PTL 1: Japanese Unexamined Patent Application Publication No. 2022-091669

### SUMMARY OF THE INVENTION

When a specific gas concentration in a measurement-object gas is measured by a gas sensor, even with the same specific gas concentration, the value of a pump current flowing through a measurement pump cell may vary due to a variation of the water concentration in the measurement-object gas. Thus, the accuracy of measurement of the specific gas concentration may be reduced due to the variation of the water concentration in the measurement-object gas.

The present invention has been devised to solve such a problem, and it is a main object to prevent reduction in the accuracy of measurement due to the variation of the water concentration in the measurement-object gas.

The present invention employs the following device to achieve the above-described object.

[1]A gas sensor of the present invention is a gas sensor comprising a sensor element, and a control device, the sensor element including: an element body that includes an oxygen-ion-conductive solid electrolyte layer, and is internally provided with a measurement-object gas flow portion that introduces a measurement-object gas and causes the measurement-object gas to flow therethrough; a measurement pump cell including an inner measurement electrode disposed in a measurement chamber of the measurement-object gas flow portion, the measurement pump cell being configured to pump oxygen from the measurement chamber to an outside of the element body; an adjustment pump cell including an inner adjustment electrode disposed in an oxygen concentration adjustment chamber located upstream of the measurement chamber in the measurement-object gas flow portion, the adjustment pump cell being configured to adjust an oxygen concentration in the oxygen concentration adjustment chamber; and a reference electrode disposed inside the element body so as to be in contact with a reference gas which serves as a reference for detection of a specific gas concentration that is a concentration of a specific gas in the measurement-object gas. The control device is configured to perform an adjustment-pump control process and a measurement-pump control process, the adjustment-pump control process for adjusting an oxygen concentration in the oxygen concentration adjustment chamber by controlling the adjustment pump cell so that an adjustment voltage that is a voltage across the reference electrode and the inner adjustment electrode reaches an adjustment voltage target value, the measurement-pump control process for pumping out oxygen from the measurement chamber by controlling the measurement pump cell so that a measurement voltage that is a voltage across the reference electrode and the inner measurement electrode reaches a measurement voltage target value, and let V1* [mV] be the adjustment voltage target value, then following Expression (1) and Expression (2) are satisfied:

$$V 1 ⋆ \leq - 25.7 * R + 436. \qquad ( 1 )$$

$$V 1 ⋆ \geq - 35.1 * R + 406.6 \qquad ( 2 )$$

where R=Lb*Lb/La,
La is a limiting current value [mA] of an adjustment pump current which flows through the adjustment pump cell when the adjustment pump cell pumps out oxygen from the oxygen concentration adjustment chamber with the sensor element exposed to the measurement-object gas which is a nitrogen-based gas with an oxygen concentration of 20.5%, a water concentration of 3%, and an NO concentration of 0 ppm, and Lb is a limiting current value [μA] of a measurement pump current which flows through the measurement pump cell when the measurement pump cell pumps out oxygen from the measurement chamber with the sensor element exposed to the measurement-object gas which is a nitrogen-based gas with an oxygen concentration of 0%, a water concentration of 3%, and an NO concentration of 500 ppm.

In this gas sensor, the control device performs the adjustment-pump control process and the measurement-pump control process. The oxygen concentration in the oxygen concentration adjustment chamber is adjusted by performing the adjustment-pump control process, then the measurement-object gas reaches the measurement chamber. Then the measurement-pump control process is performed, thereby causing the measurement pump current to flow to pump out oxygen from the measurement chamber. The specific gas concentration in the measurement-object gas can be measured based on the measurement pump current. In the gas sensor, let V1* [mV] be the adjustment voltage target value in the adjustment-pump control process, then the value of V1* satisfies the Expression (1) and Expression (2). Consequently, even if the water concentration in the measurement-object gas varies, it is unlikely that the measurement pump current varies due to the water concentration. Therefore, it is possible to prevent reduction in the accuracy of measurement of a specific gas concentration due to a variation of the water concentration in the measurement-object gas. The inventors have verified this fact by experiments.

[2] In the above-described gas sensor (the gas sensor according to [1]), Expression (3) and Expression (4) below may be satisfied. In this setting, it is possible to further prevent reduction in the accuracy of measurement of a specific gas concentration due to a variation of the water concentration in the measurement-object gas.

$$V 1 ⋆ \leq - 18.9 * R + 419.2 \qquad ( 3 )$$

$$V 1 ⋆ \geq - 16.2 * R + 403.4 \qquad ( 4 )$$

[3] In the above-described gas sensor (the gas sensor according to [1] or [2]), the oxygen concentration adjustment chamber may include a first internal cavity, and a second internal cavity provided downstream of the first internal cavity and upstream of the measurement chamber, the adjustment pump cell may include a main pump cell configured to adjust an oxygen concentration in the first internal cavity, and an auxiliary pump cell configured to adjust an oxygen concentration in the second internal cavity, the inner adjustment electrode may include an inner main pump electrode that is disposed in the first internal cavity and constitutes part of the main pump cell, and an inner auxiliary pump electrode that is disposed in the second internal cavity and constitutes part of the auxiliary pump cell, the adjustment voltage may be a voltage across the reference electrode and the inner auxiliary pump electrode, and the limiting current value La may be a limiting current value of a main pump current which flows through the main pump cell when the main pump cell pumps out oxygen from the first internal cavity with the sensor element exposed to the measurement-object gas which is a nitrogen-based gas with an oxygen concentration of 20.5%, a water concentration of 3%, and an NO concentration of 0 ppm.

[4]A method for determining an adjustment voltage target value for control of a first sensor element of the present invention, the sensor element including: an element body that includes an oxygen-ion-conductive solid electrolyte layer, and is internally provided with a measurement-object gas flow portion that introduces a measurement-object gas and causes the measurement-object gas to flow therethrough; a measurement pump cell including an inner measurement electrode disposed in a measurement chamber of the measurement-object gas flow portion, the measurement pump cell being configured to pump oxygen from the measurement chamber to an outside of the element body; an adjustment pump cell including an inner adjustment electrode disposed in an oxygen concentration adjustment chamber located upstream of the measurement chamber in the measurement-object gas flow portion, the adjustment pump cell being configured to adjust an oxygen concentration in the oxygen concentration adjustment chamber; and a reference electrode disposed inside the element body so as to be in contact with a reference gas which serves as a reference for detection of a specific gas concentration that is a concentration of a specific gas in the measurement-object gas. The adjustment voltage target value is a target value of an adjustment voltage when a specific gas concentration that is a concentration of a specific gas in the measurement-object gas is detected using the sensor element, the adjustment voltage being a voltage across the reference electrode and the inner adjustment electrode at a time of feedback control of the adjustment pump cell to adjust the oxygen concentration in the oxygen concentration adjustment chamber, the method including a step (a) of determining the adjustment voltage target value so that lower a diffusion resistance B relative to a diffusion resistance A, the adjustment voltage target value tends to be lower, where the diffusion resistance A is a diffusion resistance from an outside of the sensor element to the oxygen concentration adjustment chamber, and the diffusion resistance B is a diffusion resistance from the outside of the sensor element to the oxygen measurement chamber.

In the method for determining an adjustment voltage target value for control of a sensor element, the adjustment voltage target value is determined so that smaller the diffusion resistance B relative to the diffusion resistance A, the adjustment voltage target value tends to be lower, where the diffusion resistance A is the diffusion resistance from the outside of the sensor element to the oxygen concentration adjustment chamber, and the diffusion resistance B is the diffusion resistance from the outside of the sensor element to the measurement chamber. The inventors have found that the gas sensor has an optimal point for the adjustment voltage target value, the optimal point being capable of preventing reduction in the accuracy of measurement of a specific gas concentration due to a variation of the water concentration in the measurement-object gas. In addition, the inventors have found that such an optimal point for the adjustment voltage target value changes by the balance between the diffusion resistance A and the diffusion resistance B. More specifically, the inventors have found that the smaller the diffusion resistance B relative to the diffusion resistance A in the sensor element, the optimal point for the adjustment voltage target value tends to be lower. Thus, the adjustment voltage target value is easily determined to an optimal point or a value close to an optimal point by determining the adjustment voltage target value so that the smaller the diffusion resistance B relative to the diffusion resistance A, the adjustment voltage target value tends to be lower. Controlling the sensor element based on thus determined adjustment voltage target value facilitates prevention of reduction in the accuracy of measurement of a specific gas concentration due to a variation of the water concentration in the measurement-object gas.

[5] In the above-described method for determining an adjustment voltage target value (the method for determining an adjustment voltage target value according to [4]), the step (a) may include: a step (a1) of measuring, as a value correlated with the diffusion resistance A, a limiting current value La of an adjustment pump current which flows through the adjustment pump cell when the adjustment pump cell pumps out oxygen from the oxygen concentration adjustment chamber, and as a value correlated with the diffusion resistance B, a limiting current value Lb of a measurement pump current which flows through the measurement pump cell when the measurement pump cell pumps out oxygen from the measurement chamber; and a step (a2) of determining the adjustment voltage target value so that for the limiting current values La, Lb measured in the step (a1), greater the limiting current value Lb relative to the limiting current value La, the adjustment voltage target value tends to be lower. In this manner, instead of measuring the diffusion resistance A and the diffusion resistance B, the limiting current value La and the limiting current value Lb are measured, and the adjustment voltage target value can be determined to an optimal point or a value close to an optimal point based on the measured values.

[6] In the above-described method for determining an adjustment voltage target value (the method for determining an adjustment voltage target value according to [5]), in the step (a2), the adjustment voltage target value may be determined so that the adjustment voltage target value tends to be lower for a greater value R expressed below. The inventors have found that the value R measured using the sensor element has a linear relationship with the optimal point of the adjustment voltage target value. Thus, the adjustment voltage target value is easily determined to an optimal point or a value close to an optimal point by determining the adjustment voltage target value using the value R so that the adjustment voltage target value tends to be lower for a larger value R.

$$R = Lb ⋆ Lb / La .$$

[7] In the above-described method for determining an adjustment voltage target value (the method for determining an adjustment voltage target value according to [5] or [6]), in the step (a2), the adjustment voltage target value may be determined as a value corresponding to the limiting current values La, Lb measured in the step (a1) based on a prepared corresponding relationship in advance between the limiting current values La, Lb and the adjustment voltage target value. In this manner, an appropriate corresponding relationship is prepared in advance, for example, by conducting experiments on multiple sensor elements, thus in the step (a2), an appropriate adjustment voltage target value can be easily determined using the corresponding relationship.

[8] In the above-described method for determining an adjustment voltage target value (the method for determining an adjustment voltage target value according to any one of [5] to [7]), the oxygen concentration adjustment chamber may include a first internal cavity, and a second internal cavity provided downstream of the first internal cavity and upstream of the measurement chamber, the adjustment pump cell may include a main pump cell configured to adjust an oxygen concentration in the first internal cavity, and an auxiliary pump cell configured to adjust an oxygen concentration in the second internal cavity, the inner adjustment electrode may include an inner main pump electrode that is disposed in the first internal cavity and constitutes part of the main pump cell, and an inner auxiliary pump electrode that is disposed in the second internal cavity and constitutes part of the auxiliary pump cell, the adjustment voltage may be a voltage across the reference electrode and the inner auxiliary pump electrode, and the limiting current value La may be a limiting current value of a main pump current which flows through the main pump cell when the main pump cell pumps out oxygen from the first internal cavity.

[9]A method for determining an adjustment voltage target value for control of a second sensor element of the present invention, the sensor element including: an element body that includes an oxygen-ion-conductive solid electrolyte layer, and is internally provided with a measurement-object gas flow portion that introduces a measurement-object gas and causes the measurement-object gas to flow therethrough; a measurement pump cell including an inner measurement electrode disposed in a measurement chamber of the measurement-object gas flow portion, the measurement pump cell being configured to pump oxygen from the measurement chamber to an outside of the element body; an adjustment pump cell including an inner adjustment electrode disposed in an oxygen concentration adjustment chamber located upstream of the measurement chamber in the measurement-object gas flow portion, the adjustment pump cell being configured to adjust an oxygen concentration in the oxygen concentration adjustment chamber; and a reference electrode disposed inside the element body so as to be in contact with a reference gas which serves as a reference for detection of a specific gas concentration that is a concentration of a specific gas in the measurement-object gas. The sensor element is configured to, when a specific gas concentration that is a concentration of a specific gas in the measurement-object gas is detected, perform an adjustment-pump control process and a measurement-pump control process, the adjustment-pump control process for adjusting the oxygen concentration in the oxygen concentration adjustment chamber by controlling the adjustment pump cell so that an adjustment voltage that is a voltage across the reference electrode and the inner adjustment electrode reaches the adjustment voltage target value, the measurement-pump control process for pumping out oxygen from the measurement chamber by controlling the measurement pump cell so that a measurement voltage that is a voltage across the reference electrode and the inner measurement electrode reaches a measurement voltage target value, the method including: a step (b1) of setting the measurement voltage target value to a predetermined value, setting the adjustment voltage target value to a temporary target value, and measuring a first measurement pump current and a second measurement pump current, the first measurement pump current being caused to flow through the measurement pump cell when the adjustment-pump control process and the measurement-pump control process are performed on a first gas as the measurement-object gas, the first gas having a water concentration of a first concentration Ch1, the second measurement pump current being caused to flow through the measurement pump cell when the adjustment-pump control process and the measurement-pump control process are performed on a second gas as the measurement-object gas, the second gas having a water concentration of a second concentration Ch2 lower than the first concentration Ch 1; and a step (b2) of changing the temporary target value and performing the step (b1) for multiple times, and determining the temporary target value to be the adjustment voltage target value for control of the sensor element, the temporary target value causing a discrepancy between the first measurement pump current and the second measurement pump current to fall within a predetermined minute range.

In the method for determining an adjustment voltage target value for control of a sensor element, in the step (b1), the adjustment voltage target value is set to a temporary target value, and the first measurement pump current and the second measurement pump current are measured, which flow through when the adjustment-pump control process and the measurement-pump control process are performed using the first gas and the second gas having different water concentrations. As described above, the gas sensor element has an optimal point for the adjustment voltage target value, the optimal point being capable of preventing reduction in the accuracy of measurement of a specific gas concentration due to a variation of the water concentration in the measurement-object gas. Therefore, when the temporary target value is an optimal point for the adjustment voltage target value or a value close to an optimal point, the first measurement pump current and the second measurement pump current measured in the step (b1) have the same value or values close to each other. Thus, an appropriate adjustment voltage target value can be found by changing the temporary target value, and performing the step (b1) for multiple times. In the step (b2), change in the temporary target value and the step (b1) are performed for multiple times in this manner, and a temporary target value is determined to be the adjustment voltage target value for control of the sensor element, the temporary target value causing the discrepancy between the first measurement pump current and the second measurement pump current to fall within a predetermined minute range. Thus, the adjustment voltage target value can be determined to an optimal point or a value close to an optimal point. Controlling the sensor element based on thus determined adjustment voltage target value can prevent reduction in the accuracy of measurement of a specific gas concentration due to a variation of the water concentration in the measurement-object gas.

[10]A method for manufacturing a sensor element, including the method (the method for determining an adjustment voltage target value according to any one of [4] to [9]) for determining an adjustment voltage target value for control of the above-described sensor element, and being associated with the adjustment voltage target value, the method comprising: a manufacturing process for manufacturing the sensor element; a target value determination process for determining the adjustment voltage target value by performing, on the manufactured sensor element, the steps of the method for determining the adjustment voltage target value for control of the sensor element; and an association process for associating the manufactured sensor element with the determined adjustment voltage target value.

As with the above-described method for determining an adjustment voltage target value, with the method for manufacturing a sensor element, it is possible to determine an adjustment voltage target value which can prevent reduction in the accuracy of measurement of a specific gas concentration due to a variation of the water concentration in the measurement-object gas. Since the determined adjustment voltage target value is associated with the sensor element, when a control device is subsequently connected to the sensor element, for example, an adjustment voltage target value suitable for the sensor element can be set.

[11] The above-described method for manufacturing a sensor includes: the processes of the above-described method for manufacturing a sensor element (the method for manufacturing a sensor element according to [10]); and a connection process for connecting the sensor element associated with the adjustment voltage target value to a control device that feedback-controls the adjustment pump cell so that a voltage across the reference electrode and the inner adjustment electrode reaches the associated adjustment voltage target value.

With the method for manufacturing a gas sensor, a gas sensor including a control device and a sensor element can be manufactured. In the manufactured gas sensor, the control device controls the adjustment pump cell so that the voltage across the reference electrode and the inner adjustment electrode reaches the adjustment voltage target value determined by the above-described method for determining an adjustment voltage target value, thus it is possible to prevent reduction in the accuracy of measurement of a specific gas concentration due to a variation of the water concentration in the measurement-object gas.

### BRIEF DESCRIPTION OF THE DRAWINGS

FIG. 1 is a schematic cross-sectional view illustrating an example of a configuration of a gas sensor 100.

FIG. 2 is a block diagram illustrating an electrical connection relationship between a control device 95 and cells.

FIG. 3 is a graph illustrating a relationship between target value V1*, water concentration and pump current Ip2.

FIG. 4 is a graph illustrating a relationship between target value V1* and ΔIp2offset.

FIG. 5 is a graph illustrating a relationship between value R, target value V1* and ΔIp2offset.

FIG. 6 is a schematic cross-sectional view of a sensor element 201.

### DETAILED DESCRIPTION OF THE INVENTION

### First Embodiment

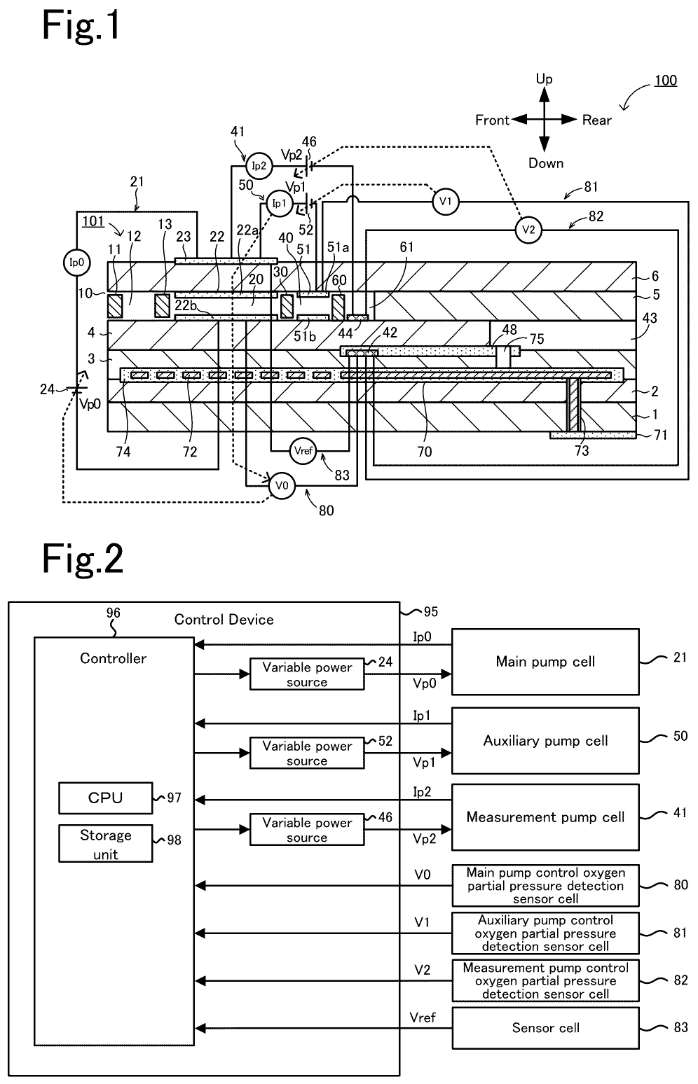

Next, an embodiment of a gas sensor of the present invention will be described using the drawings. FIG. 1 is a schematic cross-sectional view illustrating an example of a configuration of a gas sensor 100 which is an embodiment of the present invention. FIG. 2 is a block diagram illustrating an electrical connection relationship between a control device 95 and cells. The gas sensor 100 is mounted on, for example, a pipe arranged, such as an exhaust gas pipe of an internal combustion engine. The gas sensor 100 detects the concentration of a specific gas such as NOx and ammonia in a measurement-object gas which is the exhaust gas from an internal combustion engine. In the present embodiment, the gas sensor 100 measures the NOx concentration as the specific gas concentration. The gas sensor 100 includes: a sensor element 101 having an elongate rectangular parallelepiped shape; cells 21, 41, 50, 80 to 83 included in the sensor element 101; and the control device 95 having variable power sources 24, 46, 52 and configured to control the entire gas sensor 100.

The sensor element 101 is an element having a layered body in which six layers, that is, a first substrate layer 1, a second substrate layer 2, a third substrate layer 3, a first solid electrolyte layer 4, a spacer layer 5, and a second solid electrolyte layer 6, each made up of an oxygen-ion-conductive solid electrolyte layer made of zirconia (ZrO₂) or the like, are laminated in this order from a lower side in the drawing. The solid electrolyte forming these six layers is a dense, airtight one. The sensor element 101 is manufactured by, for example, applying predetermined processing, printing of a circuit pattern, and the like on a ceramic green sheet corresponding to each layer, then laminating those sheets, and further firing the sheets to be integrated.

At a tip end portion side of the sensor element 101 (left end portion side in FIG. 1), a gas inlet port 10, a first diffusion control section 11, a buffer space 12, a second diffusion control section 13, a first internal cavity 20, a third diffusion control section 30, a second internal cavity 40, a fourth diffusion control section 60, and a third internal cavity 61 are formed adjacent to each other so as to communicate with each other in this order between the under surface of the second solid electrolyte layer 6 and the top surface of the first solid electrolyte layer 4.

The gas inlet port 10, the buffer space 12, the first internal cavity 20, the second internal cavity 40, and the third internal cavity 61 are spaces of which top parts, bottom parts, and side parts, provided by hollowing the spacer layer 5, are respectively defined by the under surface of the second solid electrolyte layer 6, the top surface of the first solid electrolyte layer 4, and the side surface of the spacer layer 5 inside the sensor element 101.

Each of the first diffusion control section 11, the second diffusion control section 13, and the third diffusion control section 30 is provided as two laterally long slits (openings of which the longitudinal direction is a direction perpendicular to the drawing). The fourth diffusion control section 60 is provided as a single laterally long slit (an opening of which the longitudinal direction is a direction perpendicular to the drawing) formed as a clearance from the under surface of the second solid electrolyte layer 6. A part from the gas inlet port 10 to the third internal cavity 61 is also referred to as measurement-object gas flow portion.

At a location farther from the tip end side than the measurement-object gas flow portion, a reference gas inlet space 43 is provided between the top surface of the third substrate layer 3 and the under surface of the spacer layer 5 at a location at which the side part is defined by the side surface of the first solid electrolyte layer 4. For example, the atmosphere is introduced into the reference gas inlet space 43 as a reference gas at the time of measuring a NOx concentration.

A reference gas inlet layer 48 is a layer made of porous ceramics. The reference gas is introduced into the reference gas inlet layer 48 through the reference gas inlet space 43. The reference gas inlet layer 48 is formed so as to coat the reference electrode 42.

The reference electrode 42 is an electrode formed in such a manner in which the reference electrode 42 is sandwiched by the top surface of the third substrate layer 3 and the first solid electrolyte layer 4. As described above, the reference gas inlet layer 48 that communicates with the reference gas inlet space 43 is provided around the reference electrode 42. As will be described later, it is possible to measure an oxygen concentration (oxygen partial pressure) in the first internal cavity 20, an oxygen concentration (oxygen partial pressure) in the second internal cavity 40, and an oxygen concentration (oxygen partial pressure) in the third internal cavity 61 by using the reference electrode 42. The reference electrode 42 is formed as a porous cermet electrode (for example, a cermet electrode of Pt and ZrO₂).

In the measurement-object gas flow portion, the gas inlet port 10 is a portion that is open to an external space, and a measurement-object gas is taken into the sensor element 101 from the external space through the gas inlet port 10. The first diffusion control section 11 is a portion that applies predetermined diffusion resistance to a measurement-object gas taken in through the gas inlet port 10. The buffer space 12 is a space provided to guide the measurement-object gas introduced from the first diffusion control section 11 to the second diffusion control section 13. The second diffusion control section 13 is a portion that applies predetermined diffusion resistance to the measurement-object gas introduced from the buffer space 12 into the first internal cavity 20. When the measurement-object gas is introduced from the outside of the sensor element 101 into the first internal cavity 20, the measurement-object gas rapidly taken into the sensor element 101 through the gas inlet port 10 due to pressure fluctuations of the measurement-object gas in the external space (due to pulsation of exhaust pressure when the measurement-object gas is the exhaust gas of an automobile) is not directly introduced into the first internal cavity 20 but, after pressure fluctuations of the measurement-object gas are cancelled out through the first diffusion control section 11, the buffer space 12, and the second diffusion control section 13, the measurement-object gas is introduced into the first internal cavity 20. With this configuration, pressure fluctuations of the measurement-object gas introduced into the first internal cavity 20 are almost ignorable. The first internal cavity 20 is provided as a space used to adjust an oxygen partial pressure in the measurement-object gas introduced through the second diffusion control section 13. The oxygen partial pressure is adjusted by the operation of a main pump cell 21.

The main pump cell 21 is an electrochemical pump cell made up of an inner pump electrode 22 having a ceiling electrode portion 22a provided almost all over the under surface of the second solid electrolyte layer 6, facing the first internal cavity 20, the outer pump electrode 23 provided so as to be exposed to the external space in a region of the top surface of the second solid electrolyte layer 6, corresponding to the ceiling electrode portion 22a, and the second solid electrolyte layer 6 sandwiched by these electrodes.

The inner pump electrode 22 is formed over the upper and lower solid electrolyte layers (the second solid electrolyte layer 6 and the first solid electrolyte layer 4) defining the first internal cavity 20, and the spacer layer 5 providing a side wall. Specifically, the ceiling electrode portion 22a is formed on the under surface of the second solid electrolyte layer 6, providing a ceiling surface of the first internal cavity 20, a bottom electrode portion 22b is formed on the top surface of the first solid electrolyte layer 4, providing a bottom surface, a side electrode portion (not shown) is formed on the side wall surface (inner surface) of the spacer layer 5, making both side wall portions of the first internal cavity 20, so as to connect those ceiling electrode portion 22a and the bottom electrode portion 22b, and the inner pump electrode 22 is disposed with a structure in a tunnel form at a portion where the side electrode portion is disposed.

The inner pump electrode 22 and the outer pump electrode 23 each are formed as a porous cermet electrode (for example, a cermet electrode of Pt and ZrO₂, having an Au content of 1 percent). The inner pump electrode 22 that contacts with a measurement-object gas is formed by using a material of which the reduction ability for NOx components in the measurement-object gas is lowered.

By passing a pump current Ip0 in a positive direction or a negative direction between the inner pump electrode 22 and the outer pump electrode 23 by applying a desired pump voltage Vp0 between the inner pump electrode 22 and the outer pump electrode 23, the main pump cell 21 is capable of pumping out oxygen in the first internal cavity 20 to the external space or pumping oxygen in the external space into the first internal cavity 20.

In order to detect an oxygen concentration (oxygen partial pressure) in an atmosphere in the first internal cavity 20, an electrochemical sensor cell, that is, a main pump control oxygen partial pressure detection sensor cell 80, is made up of the inner pump electrode 22, the second solid electrolyte layer 6, the spacer layer 5, the first solid electrolyte layer 4, the third substrate layer 3, and the reference electrode 42.

An oxygen concentration (oxygen partial pressure) in the first internal cavity 20 is found by measuring an electromotive force (voltage V0) in the main pump control oxygen partial pressure detection sensor cell 80. In addition, the pump current Ip0 is controlled by executing feedback control over the pump voltage Vp0 of a variable power source 24 such that the voltage V0 becomes a target value. With this configuration, it is possible to maintain the oxygen concentration in the first internal cavity 20 at a predetermined constant value.

The third diffusion control section 30 is a portion that applies predetermined diffusion resistance to a measurement-object gas of which the oxygen concentration (oxygen partial pressure) is controlled by operation of the main pump cell 21 in the first internal cavity 20 to guide the measurement-object gas to the second internal cavity 40.

The second internal cavity 40 is provided as a space used to further adjust the oxygen partial pressure by using an auxiliary pump cell 50 for the measurement-object gas adjusted in the oxygen concentration (oxygen partial pressure) in the first internal cavity 20 in advance and then introduced through the third diffusion control section 30. With this configuration, it is possible to highly accurately maintain the oxygen concentration in the second internal cavity 40 at a constant value, so it is possible to measure a highly accurate NOx concentration with the gas sensor 100.

The auxiliary pump cell 50 is an auxiliary electrochemical pump cell made up of an auxiliary pump electrode 51 having a ceiling electrode portion 51a provided substantially all over the under surface of the second solid electrolyte layer 6, facing the second internal cavity 40, the outer pump electrode 23 (not limited to the outer pump electrode 23, and an adequate electrode outside the sensor element 101 may be used), and the second solid electrolyte layer 6.

The auxiliary pump electrode 51 is disposed in the second internal cavity 40 with a structure in a similar tunnel form to that of the inner pump electrode 22 provided in the above-described first internal cavity 20. In other words, the auxiliary pump electrode 51 has such a structure in a tunnel form that a ceiling electrode portion 51a is formed on the second solid electrolyte layer 6 providing the ceiling surface of the second internal cavity 40, a bottom electrode portion 51b is formed on the first solid electrolyte layer 4 providing the bottom surface of the second internal cavity 40, a side electrode portion (not shown) that couples those ceiling electrode portion 51a and bottom electrode portion 51b is formed on each of both wall surfaces of the spacer layer 5, providing a side wall of the second internal cavity 40. The auxiliary pump electrode 51, as well as the inner pump electrode 22, is formed by using a material of which the reduction ability for NOx components in the measurement-object gas is lowered.

By applying a desired voltage Vp1 between the auxiliary pump electrode 51 and the outer pump electrode 23, the auxiliary pump cell 50 is capable of pumping out oxygen in an atmosphere in the second internal cavity 40 to the external space or pumping oxygen from the external space into the second internal cavity 40.

In order to control an oxygen partial pressure in an atmosphere in the second internal cavity 40, an electrochemical sensor cell, that is, an auxiliary pump control oxygen partial pressure detection sensor cell 81, is made up of the auxiliary pump electrode 51, the reference electrode 42, the second solid electrolyte layer 6, the spacer layer 5, the first solid electrolyte layer 4, and the third substrate layer 3.

The auxiliary pump cell 50 performs pumping with a variable power source 52 of which the voltage is controlled in accordance with an electromotive force (voltage V1) detected by the auxiliary pump control oxygen partial pressure detection sensor cell 81. With this configuration, the oxygen partial pressure in an atmosphere in the second internal cavity 40 is controlled to a low partial pressure that substantially does not influence measurement of NOx.

Together with this, its pump current Ip1 is used to control the electromotive force of the main pump control oxygen partial pressure detection sensor cell 80. Specifically, the pump current Ip1 is input to the main pump control oxygen partial pressure detection sensor cell 80 as a control signal, and the gradient of the oxygen partial pressure in the measurement-object gas to be introduced from the third diffusion control section 30 into the second internal cavity 40 is controlled to be constantly unchanged by controlling the above-described target value of the voltage V0. When used as a NOx sensor, the oxygen concentration in the second internal cavity 40 is maintained at a constant value of about 0.001 ppm by the functions of the main pump cell 21 and auxiliary pump cell 50.

The fourth diffusion control section 60 is a portion that applies predetermined diffusion resistance to measurement-object gas of which the oxygen concentration (oxygen partial pressure) is controlled by operation of the auxiliary pump cell 50 in the second internal cavity 40 to guide the measurement-object gas to the third internal cavity 61. The fourth diffusion control section 60 plays a role in limiting the amount of NOx flowing into the third internal cavity 61.

The third internal cavity 61 is provided as a space used to perform a process related to measurement of a nitrogen oxide (NOx) concentration in a measurement-object gas on the measurement-object gas adjusted in oxygen concentration (oxygen partial pressure) in the second internal cavity 40 in advance and then introduced through the fourth diffusion control section 60. Measurement of a NOx concentration is mainly performed by operation of a measurement pump cell 41 in the third internal cavity 61.

The measurement pump cell 41 measures a NOx concentration in the measurement-object gas in the third internal cavity 61. The measurement pump cell 41 is an electrochemical pump cell made up of a measurement electrode 44 provided on the top surface of the first solid electrolyte layer 4, facing the third internal cavity 61, the outer pump electrode 23, the second solid electrolyte layer 6, the spacer layer 5, and the first solid electrolyte layer 4. The measurement electrode 44 is a porous cermet electrode made of a material of which the reduction ability for NOx components in the measurement-object gas is raised as compared to the inner pump electrode 22. The measurement electrode 44 also functions as a NOx reduction catalyst that reduces NOx present in an atmosphere in the third internal cavity 61.

The measurement pump cell 41 is capable of pumping out oxygen produced as a result of decomposition of nitrogen oxides in an atmosphere around the measurement electrode 44 and detecting the amount of oxygen produced as a pump current Ip2.

In order to detect an oxygen partial pressure around the measurement electrode 44, an electrochemical sensor cell, that is, a measurement pump control oxygen partial pressure detection sensor cell 82, is made up of the first solid electrolyte layer 4, the third substrate layer 3, the measurement electrode 44, and the reference electrode 42. A variable power source 46 is controlled in accordance with an electromotive force (voltage V2) detected by the measurement pump control oxygen partial pressure detection sensor cell 82.

A measurement-object gas guided into the second internal cavity 40 reaches the measurement electrode 44 in the third internal cavity 61 through the fourth diffusion control section 60 in a situation in which the oxygen partial pressure is controlled. Nitrogen oxides in the measurement-object gas around the measurement electrode 44 are reduced (2NO→N₂+O₂) to produce oxygen. The produced oxygen is to be pumped by the measurement pump cell 41. At this time, the voltage Vp2 of the variable power source 46 is controlled such that the voltage V2 detected by the measurement pump control oxygen partial pressure detection sensor cell 82 is constant (target value). The amount of oxygen produced around the measurement electrode 44 is proportional to the concentration of nitrogen oxides in the measurement-object gas, so a nitrogen oxide concentration in the measurement-object gas is calculated by using the pump current Ip2 in the measurement pump cell 41.

When an oxygen partial pressure detection device is constructed as an electrochemical sensor cell by combining the measurement electrode 44, the first solid electrolyte layer 4, the third substrate layer 3, and the reference electrode 42, an electromotive force according to the difference between the amount of oxygen produced by reduction of the NOx component in the atmosphere around the measurement electrode 44, and the amount of oxygen contained in the reference atmosphere can be detected, and accordingly, the concentration of the NOx component in the measurement-object gas can be determined.

An electrochemical sensor cell 83 is made up of the second solid electrolyte layer 6, the spacer layer 5, the first solid electrolyte layer 4, the third substrate layer 3, the outer pump electrode 23, and the reference electrode 42, and it is possible to detect an oxygen partial pressure in a measurement-object gas outside the sensor by using an electromotive force (voltage Vref) obtained by the sensor cell 83.

In the gas sensor 100 having such a configuration, a measurement-object gas of which the oxygen partial pressure is maintained at a constantly unchanged low value (a value that substantially does not influence measurement of NOx) is supplied to the measurement pump cell 41 by operating the main pump cell 21 and the auxiliary pump cell 50. Therefore, it is possible to find a NOx concentration in the measurement-object gas in accordance with a pump current Ip2 that flows as a result of pumping out oxygen, produced by reduction of NOx, by the measurement pump cell 41 substantially in proportion to a NOx concentration in the measurement-object gas.

In addition, the sensor element 101 includes the heater section 70 that plays a role in temperature adjustment for maintaining the temperature of the sensor element 101 by heating in order to increase the oxygen ion conductivity of the solid electrolyte. The heater section 70 includes a heater connector electrode 71, a heater 72, a through-hole 73, a heater insulating layer 74, and a pressure release hole 75.

The heater connector electrode 71 is an electrode formed in such a manner as to be in contact with the under surface of the first substrate layer 1. Connection of the heater connector electrode 71 to an external power source allows electric power to be supplied from the outside to the heater section 70.

The heater 72 is an electrical resistor which is formed to be vertically sandwiched between the second substrate layer 2 and the third substrate layer 3. The heater 72 is coupled to the heater connector electrode 71 via the through-hole 73, receives electric power supplied from the outside through the heater connector electrode 71 to generate heat, and performs heating and heat retention on the solid electrolyte constituting the sensor element 101.

The heater 72 is embedded all over the region from the first internal cavity 20 to the third internal cavity 61, and is capable of adjusting the overall sensor element 101 to a temperature at which the solid electrolyte is activated.

The heater insulating layer 74 is an electrically insulating layer formed of an insulating material, such as alumina, on the top and under surfaces of the heater 72. The heater insulating layer 74 is formed for the purpose of obtaining an electrical insulation property between the second substrate layer 2 and the heater 72 and an electrical insulation property between the third substrate layer 3 and the heater 72.

The pressure release hole 75 is a portion provided so as to extend through the third substrate layer 3 and the reference gas inlet layer 48 and communicate with the reference gas inlet space 43. The pressure release hole 75 is formed for the purpose of easing an increase in internal pressure resulting from an increase in temperature in the heater insulating layer 74.

As shown in FIG. 2, the control device 95 includes the above-mentioned variable power sources 24, 46, 52, and a controller 96. The controller 96 is a microprocessor including a CPU 97, and a storage unit 98. The storage unit 98 is a non-volatile memory capable of rewriting information, and can store various programs and various data, for example. The controller 96 receives input of voltage V0 detected by the main pump control oxygen partial pressure detection sensor cell 80, voltage V1 detected by the auxiliary pump control oxygen partial pressure detection sensor cell 81, voltage V2 detected by the measurement pump control oxygen partial pressure detection sensor cell 82, voltage Vref detected by the sensor cell 83, pump current Ip0 detected by the main pump cell 21, pump current Ip1 detected by the auxiliary pump cell 50 and pump current Ip2 detected by the measurement pump cell 41. The controller 96 controls the voltages Vp0, Vp1, Vp2 output by the variable power sources 24, 46, 52 by outputting a control signal to the variable power sources 24, 46, 52, thereby controlling the main pump cell 21, the measurement pump cell 41 and the auxiliary pump cell 50. The storage unit 98 also stores the later-described target values V0*, V1*, V2*, etc. The CPU 97 of the controller 96 controls the cells 21, 41, 50 by referring to these target values V0*, V1*, V2*.

The controller 96 executes an auxiliary pump control process of controlling the auxiliary pump cell 50 so that the oxygen concentration in the second internal cavity 40 reaches a target concentration. Specifically, the controller 96 controls the auxiliary pump cell 50 by executing feedback control on the voltage Vp1 of the variable power source 52 so that the voltage V1 reaches a constant value (referred to as target value V1*). The target value V1* is defined as the value that causes the oxygen concentration in the second internal cavity 40 to reach a predetermined low oxygen concentration that does not substantially affect measurement of NOx.

The controller 96 executes a main pump control process of controlling the main pump cell 21 so that the pump current Ip1 flowing when the oxygen concentration in the second internal cavity 40 is adjusted by the auxiliary pump cell 50 in the auxiliary pump control process reaches a target current (referred to as target value Ip1*). Specifically, the controller 96 sets (feedback-controls) a target value (referred to as a target value V0*) of the voltage V0 based on the pump current Ip1 so that the pump current Ip1 caused to flow by the voltage Vp1 reaches the constant target current Ip1*. The controller 96 then performs feedback control on the pump voltage Vp0 of the variable power source 24 so that the voltage V0 reaches the target value V0* (in other words, the oxygen concentration in the first internal cavity 20 reaches the target concentration). The gradient of oxygen partial pressure in the measurement-object gas to be introduced from the third diffusion control section 30 into the second internal cavity 40 is made unchanged constantly by the main pump control process. The target value V0* is set to a value which causes the oxygen concentration in the first internal cavity 20 to be higher than 0% and reach a low oxygen concentration. The pump current Ip0 which flows during the main pump control process varies according to the oxygen concentration in the measurement-object gas (that is, the measurement-object gas in the vicinity of the sensor element 101) which flows into the measurement-object gas flow portion through the gas inlet port 10. Thus, the controller 96 can also detect the oxygen concentration in the measurement-object gas based on the pump current Ip0.

The main pump control process and the auxiliary pump control process described above are also collectively referred as an adjustment pump control process. The first internal cavity 20 and the second internal cavity 40 are also collectively referred as an oxygen concentration adjustment chamber. The main pump cell 21 and the auxiliary pump cell 50 are also collectively referred as an adjustment pump cell. The controller 96 executes the adjustment pump control process, thus the adjustment pump cell adjusts the oxygen concentration in the oxygen concentration adjustment chamber.

In addition, the controller 96 performs a measurement-pump control process for controlling the measurement pump cell 41 so that the voltage V2 reaches a constant value (referred to as a target value V2*) (in other words, so that the oxygen concentration in the third internal cavity 61 reaches a predetermined low concentration). Specifically, the controller 96 controls the measurement pump cell 41 by feedback-controlling the voltage Vp2 of the variable power source 46 so that the voltage V2 reaches the target value V2*. Oxygen is pumped out from the third internal cavity 61 by the measurement-pump control process. The target value V2* may be e.g., 380 mV or higher and 420 mV or lower.

Execution of the measurement pump control process causes oxygen to be pumped out from the third internal cavity 61 so that the oxygen produced due to reduction of NOx in the measurement-object gas in the third internal cavity 61 become substantially zero. The controller 96 obtains a pump current Ip2 as a detected value corresponding to the oxygen produced in the third internal cavity 61 from a specific gas (herein, NOx), and calculates the NOx concentration in the measurement-object gas based on the pump current Ip2.

The storage unit 98 stores a relational expression (for example, an expression of a linear function or a quadratic function) or a map as a correspondence relationship between the pump current Ip2 and the NOx concentration. Such a relational expression or map can be determined in advance by an experiment.

An example of an NOx concentration detection process of detecting an NOx concentration in the measurement-object gas performed by the controller 96 of thus configured the gas sensor 100 will be described below. Before starting the NOx concentration detection process, the CPU 97 of the controller 96 first controls the electric power supplied to the heater section 70 by a heater power source which is not illustrated so that the temperature of the heater 72 reaches a target temperature (e.g., 800° C.). When the temperature of the heater 72 reaches close to the target temperature, the CPU 97 starts the NOx concentration detection process. In the NOx concentration detection process, the CPU 97 first starts acquisition of the respective voltages V0, V1, V2, Vref from the above-described sensor cells 80 to 83, and control of the above-described pump cells 21, 41, 50, in other words, the adjustment-pump control process and the measurement-pump control process. When the measurement-object gas is introduced through the gas inlet 10 in this state, the measurement-object gas passes through the first diffusion control section 11, the buffer space 12 and the second diffusion control section 13 in that order, and reaches the first internal cavity 20. Next, in the first internal cavity 20 and the second internal cavity 40, the oxygen concentration in the measurement-object gas is adjusted by the main pump cell 21 and the auxiliary pump cell 50, and the measurement-object gas after the adjustment reaches the third internal cavity 61. The CPU 97 then detects the NOx concentration in the measurement-object gas based on the acquired pump current Ip2 and the corresponding relationship stored in the storage unit 98. The CPU 97 transmits the value of the detected NOx concentration to an engine ECU which is not illustrated, and ends the NOx concentration detection process. The CPU 97 may perform the NOx concentration detection process e.g., at a timing for every predetermined time interval, or a timing of instructions from the engine ECU for detection of NOx concentration.

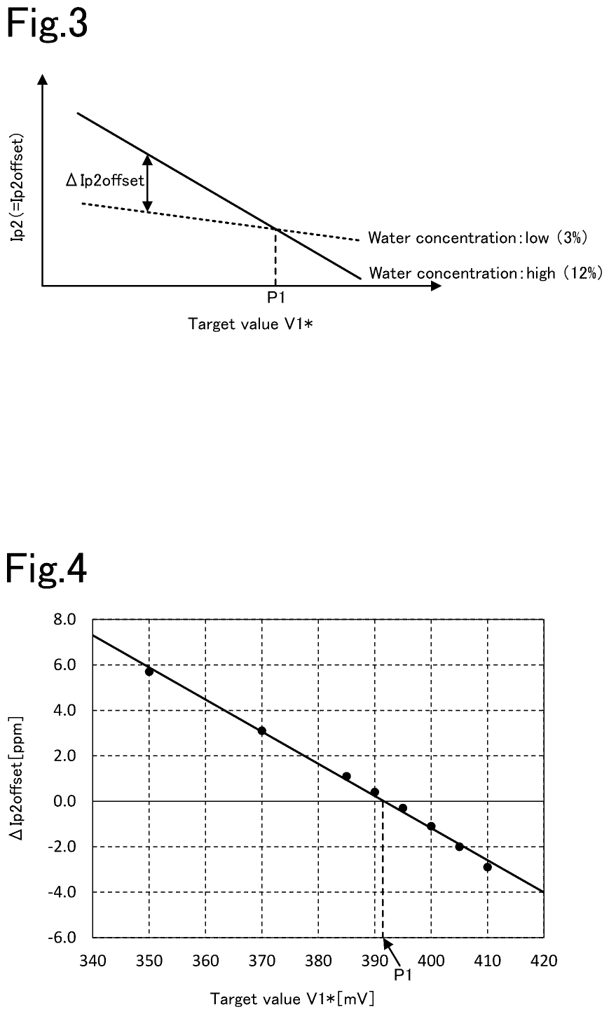

The inventors have studied the relationship between the target value V1* of the gas sensor 100, and the variation of the pump current Ip2 due to a variation of the water concentration in the measurement-object gas. As the measurement-object gas, two types of gas, that is, a first gas and a second gas are prepared, the first gas being a nitrogen-based gas with an oxygen concentration of 20.5%, a water concentration of 12%, and an NO concentration of 0 ppm, the second gas having the same composition of the first gas except for a water concentration of 3%. The target value V2* is set to 400 mV, the target value V1* is set to 350 mV, and the pump current Ip2 is measured when the controller 96 performs the above-described NOx concentration detection process on each of the first gas and the second gas. Because the first gas and the second gas each have an NO concentration of 0 ppm, the pump current Ip2 is theoretically 0 μA in either case; however, in actuality, a slight pump current Ip2 flows. Such a pump current Ip2 which flows due to a factor other than a specific gas (NOx in this case) is referred to as an offset current Ip2offset. The value obtained by subtracting the pump current Ip2 when the second gas is used as the measurement-object gas from the pump current Ip2 when the first gas is used as the measurement-object gas is calculated as the ΔIp2offset. The ΔIp2offset corresponds the amount of change (difference) in the offset current Ip2offset between 12% water concentration and 3% water concentration in the measurement-object gas. Under the same conditions as described above other than a changed target value V1*, measurement of the pump current Ip2 and calculation of the ΔIp2offset were performed. The result is shown in FIGS. 3 and 4. FIG. 3 is a graph illustrating a relationship between the target value V1*, the water concentration and the pump current Ip2. FIG. 4 is a graph illustrating a relationship between the target value V1* and the ΔIp2offset.

In FIG. 3, the relationship between the target value V1* and the pump current Ip2 when the measurement-object gas (the first gas with a water concentration of 12% in this case) with a high water concentration is used is shown by a solid line graph, and the relationship between the target value V1* and the pump current Ip2 when the measurement-object gas (the second gas with a water concentration of 3% in this case) with a low water concentration is used is shown by a dashed line graph. Since the NO concentration of the first gas and the second gas is 0 ppm as described above, the pump current Ip2 in FIG. 3 is the offset current Ip2offset. In FIG. 3, the difference between the pump current Ip2 in the solid line graph and the pump current Ip2 in the dashed line graph both corresponding to the same target value V1* is the ΔIp2offset.

FIG. 4 is a graph of the relationship between the target value V1* and the ΔIp2offset illustrated in FIG. 3. In FIG. 4, the correspondence between the target value V1* and the ΔIp2offset for each of the target values V1* at measured eight points is shown by a black dot. FIG. 4 also shows an approximate straight line which is calculated by linear approximation based on the data of the measured eight points. The ΔIp2offset along the vertical axis of FIG. 4 is shown as the value (unit is ppm) obtained by converting the above-mentioned ΔIp2offset (unit is μA) to its equivalent in terms of NO concentration using the above-mentioned corresponding relationship between the pump current Ip2 and the NOx concentration. In FIG. 4, for example when the target value V1* is set to 350 mV, the ΔIp2offset is 5.7 ppm. This result indicates that even with the same NOx concentration in the measurement-object gas, the values of NOx concentration calculated based on the pump current Ip2 for the water concentrations of 12% and 3% differ only by 5.7 ppm.

As illustrated in FIG. 3, regardless of whether the water concentration is high or low, the offset current Ip2offset tends to be smaller (negative correlation) for a higher target value V1*, and a linear relationship is observed between the target value V1* and the offset current Ip2offset. However, when the water concentration varies, the slope of the straight line showing the relationship between the target value V1* and the offset current Ip2offset varies, and the absolute value of the negative slope of the straight line is greater for a higher water concentration. Thus, as illustrated in FIG. 3, it has been found that an intersection point is present between the solid line graph and the dashed line graph, and even if the water concentration varies, the offset current Ip2offset does not vary at the intersection point. In other words, as illustrated in FIGS. 3 and 4, it has been found that value P1 of the target value V1* is present, at which the ΔIp2offset has value 0. The value P1 calculated based on the approximate straight line of FIG. 4 was approximately 392 mV. Like the value P1, the target value V1* with the ΔIp2offset of 0 ppm is referred to as an optimal point. When the target value V1* is set to an optimal point or a value close to an optimal point, even if the water concentration in the measurement-object gas varies, the offset current Ip2offset is unlikely to vary, thus it is possible to prevent reduction in the accuracy of measurement due to a variation of the water concentration. This is the knowledge newly found by the inventors. Even if the target value V1* is at an optimal point (value P1) as illustrated in FIG. 3, the value of the offset current Ip2offset may not be 0. This is because some offset currents are also generated due to a reason other than the water concentration.

The reason why there is a relationship between the target value V1* and the ΔIp2offset as illustrated in FIGS. 3 and 4 is probably as follows.

First, the relationship between the water in the measurement-object gas and the offset current Ip2offset will be described. When the adjustment-pump control process (the main-pump control process and the auxiliary pump control process in this case) is performed in the presence of water in the measurement-object gas, at least part of the water is decomposed in the vicinity of the auxiliary pump electrode 51 due to the voltage Vp1 of the variable power source 52, and hydrogen (H₂) and oxygen (O₂) are produced. The produced oxygen is pumped out from the vicinity of the auxiliary pump electrode 51, in other words, from the second internal cavity 40 by the auxiliary pump cell 50; however, at least part of the produced hydrogen reaches the third internal cavity 61. The hydrogen which has reached the third internal cavity 61 reacts with the oxygen in the third internal cavity 61 to produce water, thus the amount of oxygen pumped out from the third internal cavity 61 by the measurement-pump control process decreases, in other words, the pump current Ip2 decreases. In contrast, when the measurement-pump control process is performed in the presence of water in the measurement-object gas, at least part of the water is decomposed in the vicinity of the measurement electrode 44 in the third internal cavity 61 due to the voltage Vp2 of the variable power source 46, and hydrogen (H₂) and oxygen (O₂) are produced. Thus, the amount of oxygen pumped out from the third internal cavity 61 in the measurement-pump control process increases, in other words, the pump current Ip2 increases. In this manner, the pump current Ip2 decreases due to the hydrogen produced from water in the vicinity of the auxiliary pump electrode 51, and the pump current Ip2 increases due to the oxygen produced from water in the vicinity of the measurement electrode 44, thus the sum of the amounts of decrease and increase in the pump current Ip2 not due to NOx in the measurement-object gas appears as the offset current Ip2offset.

For a higher target value V1*, in other words, for a lower target value of the oxygen concentration in the second internal cavity 40, the voltage Vp1 is likely to be a high value by the auxiliary-pump control process, thus in the vicinity of the auxiliary pump electrode 51, the amount of hydrogen produced by decomposition of water by the voltage Vp1 increases, thus the amount of decrease in the pump current Ip2 also increases. Thus, with a target value V1* cancelling out the amounts of decrease and increase in the pump current Ip2 due to the above-mentioned decomposition of water in the measurement-object gas, the offset current Ip2offset due to the decomposition of water is probably zero. When the offset current Ip2offset due to the decomposition of water is zero, even if the water concentration varies, the offset current Ip2offset does not vary, thus the ΔIp2offset is 0 μA (=0 ppm). Because of this reason, when the target value V1* is the value P1, the ΔIp2offset is 0 ppm in FIG. 4. When the target value V1* is lower than the value P1, the amount of increase in the pump current Ip2 due to the decomposition of water is greater than the amount of decrease in the pump current Ip2, thus the offset current Ip2offset due to decomposition of water probably has a positive value. Thus, when the target value V1* is lower than the value P1, as illustrated in FIG. 3, the pump current Ip2 is greater for a higher water concentration, and the ΔIp2offset probably has a positive value as illustrated in FIG. 4. In contrast, when the target value V1* is higher than the value P1, the amount of decrease in the pump current Ip2 due to the decomposition of water is greater than the amount of increase in the pump current Ip2, thus the offset current Ip2offset due to the decomposition of water probably has a negative value. Thus, when the target value V1* is higher than the value P1, as illustrated in FIG. 3, the pump current Ip2 is lower for a higher water concentration, and the ΔIp2offset probably has a negative value as illustrated in FIG. 4.

In addition, the inventors have found that an adjustment voltage target value (an optimal point or a value close to an optimal point of the target value V1* in this case) capable of preventing reduction of the accuracy of measurement due to a variation of the water concentration changes by the balance between a diffusion resistance A and a diffusion resistance B, the diffusion resistance A being the diffusion resistance from the outside of the sensor element 101 to the oxygen concentration adjustment chamber, the diffusion resistance B being the diffusion resistance from the outside of the sensor element 101 to the measurement chamber (the third internal cavity 61 in this case). More specifically, the inventors have found that the smaller the diffusion resistance B relative to the diffusion resistance A in the sensor element 101, the optimal point for the target value V1* tends to be lower. The reason is probably as follows. Not all the hydrogen produced by the decomposition of water in the vicinity of the auxiliary pump electrode 51 reaches the third internal cavity 61 downstream of the auxiliary pump electrode 51, and part of the hydrogen moves upstream of the auxiliary pump electrode 51, and diffuses from the gas inlet 10 to the outside of the sensor element 101. Thus, the proportion of degree of downstream and upstream movement (diffusion) of the hydrogen produced in the vicinity of the auxiliary pump electrode 51 relative to the measurement-object gas flow portion is changed by the diffusion resistance upstream and the diffusion resistance downstream of the auxiliary pump electrode 51. The diffusion resistance upstream of the auxiliary pump electrode 51 is correlated with the diffusion resistance A, and the composite resistance of the diffusion resistance upstream and the diffusion resistance downstream of the auxiliary pump electrode 51 is correlated with the diffusion resistance B. In the sensor element 101 with a lower diffusion resistance B relative to the diffusion resistance A, the hydrogen produced in the vicinity of the auxiliary pump electrode 51 is more likely to move downstream, thus the amount of decrease in the pump current Ip2 due to the hydrogen also increases. For example, when two types of sensor element 101 having the same diffusion resistance A and different diffusion resistances B are compared, in the sensor element 101 with a lower diffusion resistance B, the hydrogen produced in the vicinity of the auxiliary pump electrode 51 is more likely to move downstream, thus the amount of decrease in the pump current Ip2 due to the hydrogen also increases. Thus, in the sensor element 101, even with the same value of the target value V1*, the smaller the diffusion resistance B relative to the diffusion resistance A, the amount of decrease in the pump current Ip2 increases. As a result, it is probable that the smaller the diffusion resistance B relative to the diffusion resistance A in the sensor element 101, the optimal point (the target value V1* that can cancel out the amount of increase in the pump current Ip2 due to the decomposition of water in the vicinity of the measurement electrode 44) of the target value V1* tends to be lower. The inventors have verified this fact in the following manner.

For the sensor element 101 used for measurement in FIGS. 3 and 4, the inventors measured, as a value correlated with the diffusion resistance A, a limiting current value La of the adjustment pump current which flows through the adjustment pump cell when the adjustment pump cell pumps out oxygen from the oxygen concentration adjustment chamber. More specifically, as the limiting current value La, the limiting current value of the pump current lp0 was measured, which flows through the main pump cell 21 when the main pump cell 21 pumps out oxygen from the first internal cavity 20. Specifically, let the diffusion resistance A be the diffusion resistance (more specifically, the composite resistance of the first diffusion control section 11 and the second diffusion control section 13) from the outside of the sensor element 101 to the first internal cavity 20, and the limiting current value of the pump current lp0 was measured as the limiting current value La correlated with the diffusion resistance A. The flow rate per unit time of the measurement-object gas which is introduced from the outside of the sensor element 101 to the measurement-object gas flow portion through the gas inlet 10, and reaches the first internal cavity 20, is negatively correlated with the diffusion resistance A upstream of the first internal cavity 20, and the limiting current value La is positively correlated with the flow rate. Therefore, a negative correlation is observed between the diffusion resistance A and the limiting current value La, and for example, the limiting current value La tends to be a greater value for a lower diffusion resistance A.

For the sensor element 101 used for measurement in FIG. 3, the inventors measured, as a value correlated with the diffusion resistance B, the limiting current value Lb of the measurement pump current which flows through the measurement pump cell when the measurement pump cell pumps out oxygen from the measurement chamber. More specifically, as the limiting current value Lb, the limiting current value of the pump current Ip2 was measured, which flows through the measurement pump cell 41 when the measurement pump cell 41 pumps out oxygen from the third internal cavity 61. In other words, let the diffusion resistance B be the diffusion resistance (more specifically, the composite resistance of the first diffusion control section 11, the second diffusion control section 13, the third diffusion control section 30, and fourth diffusion control section 60) from the outside of the sensor element 101 to the third internal cavity 61, and the limiting current value of the pump current Ip2 was measured as the limiting current value Lb correlated with the diffusion resistance B. The flow rate per unit time of the measurement-object gas which is introduced from the outside of the sensor element 101 to the measurement-object gas flow portion through the gas inlet 10, and reaches the third internal cavity 61, is negatively correlated with the diffusion resistance B upstream of the third internal cavity 61, and the limiting current value Lb is positively correlated with the flow rate. Therefore, a negative correlation is observed between the diffusion resistance B and the limiting current value Lb, and for example, the limiting current value Lb tends to be a greater value for a lower diffusion resistance B.

The limiting current value La was measured in the following manner. First, as the measurement-object gas, a nitrogen-based gas (a gas having the same composition as that of the above-mentioned second gas at the time of measurement in FIGS. 3 and 4) with an oxygen concentration of 20.5%, a water concentration of 3%, and an NO concentration of 0 ppm was prepared. With the sensor element 101 exposed to the measurement-object gas, the heater 72 was energized to heat the sensor element 101 up to a predetermined target temperature (e.g., 800° C.). With no voltage applied to each of the variable power sources 46, 52, the measurement pump cell 41 and the auxiliary pump cell 50 were prohibited to be operated. In this state, the voltage Vp0 is applied across the inner pump electrode 22 and the outer pump electrode 23 by the variable power source 24 so that oxygen is pumped out from the vicinity of the inner pump electrode 22 to the vicinity of the outer pump electrode 23, and the pump current lp0 which flows between both electrodes 22, 23 was measured. Subsequently, when the voltage Vp0 is gradually increased, the pump current Ip0 is also gradually increased; however, with the voltage Vp0 higher than a certain level applied, even if the voltage Vp0 is increased, the pump current Ip0 is not increased, and reaches an upper limit. The upper limit of the pump current Ip0 was measured as the limiting current value La.

The limiting current value Lb was measured in the following manner. First, as the measurement-object gas, a nitrogen-based gas with an oxygen concentration of 0%, a water concentration of 3%, and an NO concentration of 500 ppm was prepared. With the sensor element 101 exposed to the measurement-object gas, the heater 72 was energized to heat the sensor element 101 up to a predetermined target temperature (e.g., 800° C.). With no voltage applied to each of the variable power sources 24, 52, the main pump cell 21 and the auxiliary pump cell 50 were prohibited to be operated. In this state, the voltage Vp2 is applied across the measurement electrode 44 and the outer pump electrode 23 by the variable power source 46 so that oxygen is pumped out from the vicinity of the measurement electrode 44 to the vicinity of the outer pump electrode 23, and the pump current Ip2 which flows between both electrodes 44, 23 was measured. Subsequently, when the voltage Vp2 is gradually increased, the pump current Ip2 is also gradually increased; however, with the voltage Vp2 higher than a certain level applied, even if the voltage Vp2 is increased, the pump current Ip2 is not increased, and reaches an upper limit. The upper limit of the pump current Ip2 was measured as the limiting current value Lb.

As a value representing the balance between the diffusion resistances A, B, the inventors devised the use of the value R calculated by the expression, R=Lb*Lb/La in terms of the limiting current values La, Lb. As seen from the above expression, the value R is obtained by dividing the square of the limiting current value Lb by the limiting current value La. The larger the value R, the limiting current value Lb is greater relative to the limiting current value La, which indicates that the diffusion resistance B is lower relative to the diffusion resistance A. When the value R is calculated, the limiting current value La is expressed in the unit of [mA], and the limiting current value Lb is expressed in the unit of [μA]. For the sensor element 101 used for the above-mentioned measurement in FIGS. 3 and 4, the value R was 0.998 (unit is [μA²/mA]).

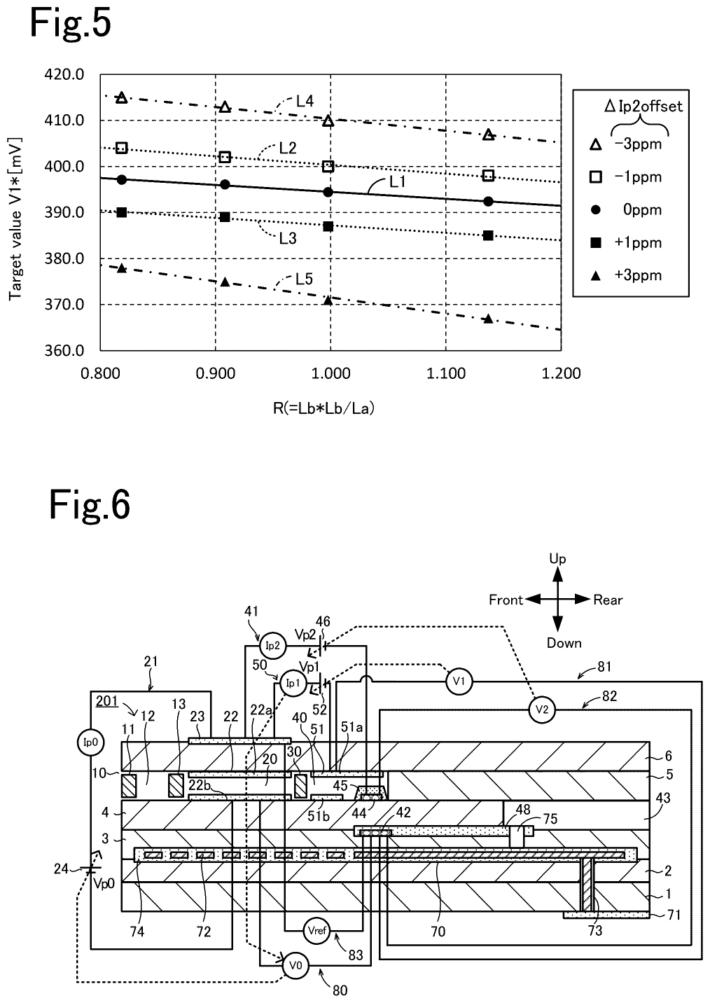

In addition to the sensor element 101 used for the measurement in FIGS. 3 and 4, four sensor elements 101 having the same value R of 0.998 as in the above sensor element 101 were prepared. For the four sensor elements 101, the relationship between the target value V1* and the ΔIp2offset was studied by the same method used for acquisition of data in FIGS. 3 and 4. In the same manner as the calculation of an approximate straight line in FIG. 4, an approximate straight line was calculated by linear approximation based on the relationship between the target value V1* and the ΔIp2offset for these five sensor elements 101 in total. The target value V1* was calculated for each case where the ΔIp2offset is 0 ppm, −1 ppm, +1 ppm, −3 ppm, or +3 ppm based on the approximate straight line. Similarly, three types of sensor element 101 having the value R of 0.818, 0.908, 1.137 were prepared with each type consisting of five sensor elements 101. For these sensor elements 101, the value R was adjusted to the above-mentioned values by changing the shape of the gap (slit) between the second diffusion control section 13 and the fourth diffusion control section 60. For the five sensor elements 101 having the same value R, the target value V1* was calculated, which causes the ΔIp2offset to be 0 ppm, −1 ppm, +1 ppm, −3 ppm, or +3 ppm by the same method described above. The result is shown in FIG. 5 and Table 1.

TABLE 1

| | V1*[mV] | | | | |
|---|---|---|---|---|---|
| | ΔIp2off- | ΔIp2off- | ΔIp2off- | ΔIp2off- | ΔIp2off- |
| R | set = 0 ppm | set = −1 ppm | set = +1 ppm | set = −3 ppm | set = +3 ppm |
| 0.818 | 397.1 | 404 | 390 | 415 | 378 |
| 0.908 | 396.1 | 402 | 389 | 413 | 375 |
| 0.998 | 394.4 | 400 | 387 | 410 | 371 |
| 1.137 | 392.4 | 398 | 385 | 407 | 367 |

As seen from the data of four points plotted as black dots in FIG. 5, it has been verified that a linear relationship is observed between the value R and the target value V1* (in other words, the optimal point of the target value V1*) for which the ΔIp2offset is 0 ppm, and the larger the value R, the optimal point of the target value V1* tends to be lower in the sensor element 101. A large value R indicates that in the sensor element 101, the limiting current value Lb is relatively larger with respect to the limiting current value La, and eventually, the diffusion resistance B is relatively lower with respect to the diffusion resistance A. Thus, as described above, it has been verified from FIG. 5 that in the sensor element 101 with the diffusion resistance B lower than the diffusion resistance A, the optimal point of the target value V1* tends to be lower. It has been verified that not only the optimal point of the target value V1*, but also the target value V1* with the ΔIp2offset relatively close to 0 ppm such as −1 ppm, +1 ppm, −3 ppm, and +3 ppm has a linear relationship with the value R. This is the knowledge newly found by the inventors. FIG. 5 shows, as a straight line L1, an approximate straight line calculated by linear approximation based on the relationship (the data of four points indicated by black dots) between the value R and the target value V1* for which the ΔIp2offset is 0 ppm. Similarly, FIG. 5 shows, as straight lines L2 to L5, approximate straight lines each indicating a relationship between the value R and the target value V1* for which the ΔIp2offset is −1 ppm, +1 ppm, −3 ppm, or +3 ppm. The linear functions for the straight lines L1 to L5 are given by the following Expressions (a) to (e).

$$Straight line L 1 : V 1 ⋆ = - 15.1 * R + 409.6 \qquad ( a )$$

$$Straight line L 2 : V 1 ⋆ = - 18.9 * R + 41 9.2 \qquad ( b )$$

$$Straight line L 3 : V 1 ⋆ = - 16.2 * R + 40 3.4 \qquad ( c )$$

$$Straight line L 4 : V 1 ⋆ = - 25.7 * R + 43 6. \qquad ( d )$$

$$Straight line L 5 : V 1 ⋆ = - 35.1 * R + 406.6 \qquad ( e )$$

From these results, it is found that in the gas sensor 100, when the points obtained by plotting the correspondence between the value R and the target value V1* of the sensor element 101 in FIG. 5 are located between the straight line L4 and the straight line L5, in other words, when the points satisfy Expression (1) and Expression (2) below, the ΔIp2offset can have a value relatively close to 0 ppm, such as a value within ±3 ppm in the gas sensor 100. Thus, in the gas sensor 100 satisfying Expression (1) and Expression (2) below, even if the water concentration in the measurement-object gas varies, the offset current Ip2offset is unlikely to vary, thus it is possible to prevent reduction in the accuracy of measurement due to a variation of the water concentration. The coefficients (slope and intercept) of Expression (1) and Expression (2) below are the same as those of Expression (d) and Expression (e) above. Also, it is found that in the gas sensor 100, when the points obtained by plotting the correspondence between the value R and the target value V1* of the sensor element 101 in FIG. 5 are located between the straight line L2 and the straight line L3, in other words, when the points satisfy Expression (3) and Expression (4) below, the ΔIp2offset can have a value further closer to 0 ppm, such as a value within ±1 ppm in the gas sensor 100. Thus, the gas sensor 100 preferably satisfies Expression (3) and Expression (4) below, thereby making it possible to further prevent reduction in the accuracy of measurement due to a variation of the water concentration. In addition, it is found that in the gas sensor 100, when the points obtained by plotting the correspondence between the value R and the target value V1* of the sensor element 101 in FIG. 5 are located on the straight line L1, in other words, when the points satisfy Expression (a) above, the target value V1* is the optimal point, and the ΔIp2offset is substantially 0 ppm, thus reduction of the accuracy of measurement due to a variation of the water concentration does not substantially occur. Therefore, in the gas sensor 100, the points obtained by plotting the correspondence between the value R and the target value V1* of the sensor element 101 in FIG. 5 are preferably close to the straight line L1 as much as possible, and more preferably located on the straight line L1.

$$V 1 ⋆ \leq - 25.7 * R + 436. \qquad ( 1 )$$

$$V 1 ⋆ \geq - 35.1 * R + 406.6 \qquad ( 2 )$$

$$V 1 ⋆ \leq - 18.9 * R + 419.2 \qquad ( 3 )$$

$$V 1 ⋆ \geq - 16.2 * R + 403.4 \qquad ( 4 )$$

In the present embodiment, in the gas sensor 100, the target value V1* is determined based on the measured value R for the sensor element 101 so that the corresponding points between the value R and the target value V1* of the sensor element 101 are located on the straight line L1, in other words, so that the corresponding points satisfy Expression (a) above, and the determined target value V1* is stored in the storage unit 98.

The corresponding relationship between the components of the present embodiment and the components of the present invention will now be clarified. A layered body in which six layers, that is, the first substrate layer 1, the second substrate layer 2, the third substrate layer 3, the first solid electrolyte layer 4, the spacer layer 5, and the second solid electrolyte layer 6 of the present embodiment are layered in this order corresponds to an element body of the present invention; the third internal cavity 61 corresponds to a measurement chamber; the measurement electrode 44 corresponds to an inner measurement electrode; the measurement pump cell 41 corresponds to a measurement pump cell; the first internal cavity 20 and the second internal cavity 40 correspond to an oxygen concentration adjustment chamber; the inner pump electrode 22 and the auxiliary pump electrode 51 correspond to an inner adjustment electrode; the main pump cell 21 and the auxiliary pump cell 50 correspond to an adjustment pump cell; the reference electrode 42 corresponds to a reference electrode; the voltage V1 corresponds to an adjustment voltage, the target value V1* corresponds to an adjustment voltage target value; the voltage V2 corresponds to a measurement voltage; the target value V2* corresponds to a measurement voltage target value; the control device 95 corresponds to a control device; the pump current lp0 corresponds to an adjustment pump current and a main pump current; and the pump current Ip2 corresponds to a measurement pump current. In addition, the inner pump electrode 22 corresponds to an inner main pump electrode, and the auxiliary pump electrode 51 corresponds to an inner auxiliary pump electrode.

In the gas sensor 100 of the present embodiment described in detail above, the adjustment voltage target value (the target value V1* in this case) in the adjustment-pump control process satisfies Expression (1) and Expression (2) stated above. Thus, even if the water concentration in the measurement-object gas varies the pump current Ip2 is unlikely to vary, thus it is possible to prevent reduction in the accuracy of measurement due to a variation of the water concentration in the measurement-object gas.

Also, with the above Expression (3) and Expression (4) satisfied by the adjustment voltage target value (the target value V1* in this case), it is possible to further prevent reduction in the accuracy of measurement due to a variation of the water concentration in the measurement-object gas.

### Second Embodiment

An embodiment of a method for manufacturing a gas sensor, including the method for determining an adjustment voltage target value for control of a sensor element of the present invention will be described. A method for manufacturing the gas sensor 100 illustrated in FIGS. 1 and 2 will be described below. A method for manufacturing the gas sensor 100 of the present embodiment includes: a manufacturing process for manufacturing the sensor element 101; a target value determination process for determining a target value V1* (an example of an adjustment voltage target value) for control of the manufactured sensor element 101; an association process for associating the manufactured sensor element 101 with the determined target value V1*; and a connection process for connecting the sensor element 101 associated with the target value V1* to the control device 95 that feedback-controls the auxiliary pump cell 50 (an example of the adjustment pump cell) so that the voltage V1 across the reference electrode 42 and the auxiliary pump electrode 51 (an example of the inner adjustment electrode, and an example of the inner auxiliary pump electrode) reaches the associated target value V1*.

In the manufacturing process, the sensor element 101 is manufactured in the following manner, for example. First, six unfired ceramic green sheets containing, as a ceramic component, an oxygen-ion-conductive solid electrolyte such as zirconia are prepared. Multiple sheet holes and necessary through-holes and the like used for positioning at the time of printing or at the time of layering are formed in the green sheets in advance. Also, a green sheet that becomes the spacer layer 5 is provided with a space by a punching process or the like in advance, the space to become the measurement-object gas flow portion. A pattern printing process and a drying process for forming various patterns on the ceramic green sheets are performed corresponding to the first substrate layer 1, the second substrate layer 2, the third substrate layer 3, the first solid electrolyte layer 4, the spacer layer 5, and the second solid electrolyte layer 6. Specifically, the formed patterns are, for example, the above-mentioned electrodes, lead wires connected to the electrodes, the reference gas inlet layer 48, and the heater section 70. The pattern printing is performed by applying paste for pattern formation to the green sheets using a publicly known screen printing technique, the paste for pattern formation being prepared according to the characteristics required for respective objects to be formed. The drying process is also performed using a publicly known drying device. When the pattern printing and drying are completed, a printing and drying process is performed on paste for bonding for layering and bonding the green sheets corresponding to the layers. The green sheets with the paste for bonding formed undergo positioning with sheet holes, and are layered in a predetermined order, then a pressure bonding process is performed by applying predetermined temperature and pressure conditions, thereby producing one layered body. Thus obtained layered body includes a plurality of sensor elements 101. The layered body is cut into pieces each having the size of the sensor element 101. The cut pieces of the layered body are fired at a predetermined firing temperature to obtain the sensor elements 101.

Note that the first to fourth diffusion control sections 11, 13, 30, 60 can be formed by printing a paste composed of a vanishing material (e.g., theobromine) on the upper and lower surfaces of the green sheet that becomes the spacer layer 5 at the time of pattern printing process, and vanishing the vanishing material at the time of firing the layered body.

Next, a target value determination process for determining a target value V1* is performed on each of multiple manufactured sensor elements 101. The target value determination process includes a step (a) of determining the target value V1* (an example of the adjustment voltage target value) so that the lower a diffusion resistance B relative to a diffusion resistance A, the target value V1* tends to be lower, where the diffusion resistance A is a diffusion resistance from the outside of the sensor element 101 to the first internal cavity 20 (an example of the oxygen concentration adjustment chamber), and the diffusion resistance B is a diffusion resistance from the outside of the sensor element 101 to the third internal cavity 61 (an example of the measurement chamber).

As described in the first embodiment, the target value V1* (an optimal point or a value close to an optimal point of the target value V1*) capable of preventing reduction of the accuracy of measurement due to a variation of the water concentration changes by the balance between the diffusion resistance A and the diffusion resistance B of the sensor element 101. Thus, in the sensor element 101 as the object for determination of the target value V1*, the target value V1* is easily determined to an optimal point or a value close to an optimal point by determining the target value V1* so that the smaller the diffusion resistance B relative to the diffusion resistance A, the target value V1* tends to be lower. Controlling the sensor element based on thus determined target value V1* can prevent reduction in the accuracy of measurement of a specific gas concentration due to a variation of the water concentration in the measurement-object gas. In the step (a), the target value V1* may be determined as the value corresponding to the diffusion resistances A, B of the sensor element 101 as the object for determination of the target value V1* based on a prepared corresponding relationship in advance between the diffusion resistances A, B and the target value V1*.

The diffusion resistance A of the sensor element 101 can be calculated based on e.g., the dimensions (the dimensions of the gap, that is, the slit) of the diffusion control sections present inclusively between the outside of the sensor element 101 and the oxygen concentration adjustment chamber. The diffusion resistance can be calculated, for example, by dividing the length (the length in the front-back direction in this case) of a diffusion control section in the flow direction of the measurement-object gas by the cross-sectional area perpendicular to the flow direction. Similarly, the diffusion resistance B can be calculated based on e.g., the dimensions of the diffusion control sections present inclusively between the outside of the sensor element 101 and the measurement chamber. The diffusion resistances A, B may be calculated by measuring the dimensions of the diffusion control sections of the manufactured sensor element 101, or calculated based on the design value of the dimensions of the diffusion control sections when the sensor element 101 is manufactured. The dimensions of the diffusion control sections are adjustable by adjusting the pattern shape at the time of printing of the above-mentioned paste composed of a vanishing material, or adjusting the thickness of the paste after printing by adjusting the viscosity of the paste. Even when the values of the diffusion resistances A, B themselves cannot be calculated, the step (a) can be performed based on the balance between the diffusion resistance A and the diffusion resistance B. For example, when two types of the sensor element 101 having the same diffusion resistance A and different diffusion resistances B are provided on determining the target values V1* (an example of the adjustment voltage target value) of multiple sensor elements 101, for the sensor element 101 having a lower diffusion resistance B, the target value V1* may be determined to be a smaller value. In this case, as compared to when target values V1* for multiple sensor elements 101 are determined to be the same value uniformly regardless of the balance between diffusion resistance A and diffusion resistance B, the target value V1* is likely to be an optimal point or a value close to an optimal point. In the present embodiment, the composite resistance of the first diffusion control section 11 and the second diffusion control section 13 which are located upstream of the first internal cavity 20 of the measurement-object gas flow portion corresponds to the diffusion resistance A. The composite resistance of the first diffusion control section 11, the second diffusion control section 13, the third diffusion control section 30, and fourth diffusion control section 60 which are located upstream of the third internal cavity 61 of the measurement-object gas flow portion corresponds to the diffusion resistance B.

The target value V1* may be determined based on a value correlated with the diffusion resistances A, B instead of based on the diffusion resistances A, B. For example, the above-described step (a) may include: a step (a1) of measuring, as a value correlated with the diffusion resistance A, the limiting current value La of the pump current Ip0 (an example of the adjustment pump current) which flows through the main pump cell 21 (an example of the adjustment pump cell) when the main pump cell 21 pumps out oxygen from the first internal cavity 20 (an example of the oxygen concentration adjustment chamber), and as a value correlated with the diffusion resistance B, the limiting current value Lb of the pump current Ip2 (the measurement pump current) which flows through the measurement pump cell 41 when the measurement pump cell 41 pumps out oxygen from the third internal cavity 61 (an example of the measurement chamber); and a step (a2) of determining the target value V1* so that for the limiting current values La, Lb measured in the step (a1), the greater the limiting current value Lb relative to the limiting current value La, the target value V1* tends to be lower.

As described in the first embodiment, a negative correlation is observed between the diffusion resistance A and the limiting current value La, and a negative correlation is observed between the diffusion resistance B and the limiting current value Lb. Thus, the step (a1) and the step (a2) are performed to measure the limiting current values La, Lb instead of the diffusion resistances A, B, and the target value V1* can be determined to an optimal point or a value close to an optimal point based on the measured values. In the manufactured sensor element 101, the limiting current values La, Lb can be measured non-destructively for the sensor element 101. Thus, for example, as compared to when the diffusion resistances A, B calculated based on the design value are used, in the step (a1), the limiting current values La, Lb can be obtained as values correlated with the actual diffusion resistances A, B which include an individual difference such as a manufacturing error of the sensor element 101. Thus, the target value V1* can be determined to be a more appropriate value.

The limiting current values La, Lb can be measured in the same manner as in the first embodiment. However, in the first embodiment, the measurement-object gas used to measure the limiting current value La is a nitrogen-based gas with an oxygen concentration of 20.5%, a water concentration of 3%, and an NO concentration of 0 ppm, and the measurement-object gas used to measure the limiting current value Lb is a nitrogen-based gas with an oxygen concentration of 0%, a water concentration of 3%, and an NO concentration of 500 ppm; however, without being limited to this, the limiting current values La, Lb may be measured using a gas with another composition. For example, the oxygen concentration of the measurement-object gas at the time of measurement of the limiting current value La is not limited to 20.5%, and it is sufficient that the measurement-object gas contain oxygen with a concentration which causes the pump current lp0 to be a limiting current. The measurement-object gas at the time of measurement of the limiting current value Lb may contain oxygen or oxide (e.g., NO) with a concentration which causes the pump current Ip2 to be a limiting current. Thus, the oxygen concentration of the measurement-object gas at the time of measurement of the limiting current value Lb is not limited to 0%, and the measurement-object gas may contain oxygen. The NO concentration of the measurement-object gas at the time of measurement of the limiting current value La is not limited to 0 ppm, and may be a value greater than 0 ppm. The NO concentration of the measurement-object gas at the time of measurement of the limiting current value Lb is not limited to 500 ppm, and may be another value, or may be 0 ppm. When the measurement-object gas contains oxygen, even if the NO concentration is 0 ppm, the limiting current value Lb can be measured. The NO concentration of the measurement-object gas may be the same both at the time of measurement of the limiting current value La and at the time of measurement of the limiting current value Lb. The water concentration of the measurement-object gas at the time of measurement of the limiting current values La, Lb is not limited to 3%, and may be another value. However, the water concentration of the measurement-object gas is preferably the same both at the time of measurement of the limiting current value La and at the time of measurement of the limiting current value Lb. The measurement-object gas at the time of measurement of the limiting current value La, and the measurement-object gas at the time of measurement of the limiting current value Lb may be the same gas. In the first embodiment, at the time of measurement of the limiting current value La, the measurement pump cell 41 and the auxiliary pump cell 50 are prohibited to be operated, but may be operated. In the first embodiment, at the time of measurement of the limiting current value Lb, the main pump cell 21 and the auxiliary pump cell 50 are prohibited to be operated, but may be operated. When the measurement-object gas at the time of measurement of the limiting current value Lb contains oxygen, the main pump cell 21 and the auxiliary pump cell 50 are preferably operated.

In the above-described step (a2), the target value V1* may be determined so that the greater the above-mentioned value R (=Lb*Lb/La), the target value V1* tends to be lower. As described in the first embodiment, the value R measured using the sensor element 101 has a linear relationship with the optimal point of the target value V1*. Thus, the target value V1* is easily determined to an optimal point or a value close to an optimal point by determining the target value V1* using the value R so that the target value V1* tends to be lower for a larger value R.

In the step (a2), the target value V1* may be determined as a value corresponding to the limiting current values La, Lb measured in the step (a1) based on a prepared corresponding relationship in advance between the limiting current values La, Lb and the target value V1*. In this manner, an appropriate corresponding relationship is prepared in advance, for example, by conducting experiments on multiple sensor elements 101, thus in the step (a2), an appropriate target value V1* can be easily determined using the corresponding relationship. For example, Expression (1) to Expression (4) and Expression (a) to Expression (e) described in the first embodiment are each a corresponding relationship between the value R and preferred target value V1*, thus an example of prepared corresponding relationship in advance between the limiting current values La, Lb and the target value V1*. Thus, for example, in the step (a2), the target value V1* corresponding to the limiting current values La, Lb measured in the step (a1) can be determined using Expression (1) and Expression (2) as the prepared corresponding relationships in advance. Specifically, the value R calculated from the limiting current values La, Lb is substituted into Expression (1) and Expression (2) to calculate the numerical range of the target value V1* satisfying Expression (1) and Expression (2), and the target value V1* may be determined as the value in the numerical range. In this manner, the target value V1* satisfying Expression (1) and Expression (2), in other words, the target value V1* such that the above-mentioned the ΔIp2offset is within ±3 ppm can be determined. As the prepared corresponding relationship in advance, Expression (3) and Expression (4) may be used, or Expression (a) may be used.

Note that when a different method for measuring the limiting current values La, Lb is adopted, the corresponding relationship between the limiting current values La, Lb and a preferred target value V1* may also change, thus the corresponding relationship is not limited to the above Expression (1) to Expression (4) and Expression (a) to Expression (e). The limiting current values La, Lb may be measured under the same condition at the time of preparation of a corresponding relationship in advance and at the time of execution of the step (a1).

When the target value V1* is determined in the target value determination process, an association process is performed to associate the determined value with the sensor element 101 which is manufactured in the manufacturing process and serves as the object for determination of the target value V1*. In other words, in each of the sensor elements 101 manufactured in the manufacturing process, the determined target value V1* is made to be identified. Direct association may be made, for example, by transporting, along with the sensor element 101, paper or the like on which the value of the target value V1* is written. Alternatively, indirect association may be made, for example, by storing, in a storage medium, a corresponding relationship between the manufacturing number assigned to the sensor element 101 and the value of the target value V1*, or managing traceability of the manufacturing process as online data by e.g., a server.

Subsequently, in the connection process, the control device 95 is connected to the sensor element 101. The value of the target value V1* associated with the sensor element 101 in the association process is stored in the storage unit 98. Thus, the control device 95 can feedback-control the auxiliary pump cell 50 so that the voltage V1 across the reference electrode 42 and the auxiliary pump electrode 51 reaches the target value V1* determined in the target value determination process. In the connection process, connecting the control device 95 to the sensor element 101 and storing the value of the target value V1* in the storage unit 98 may be performed in any order. Thereby, the gas sensor 100 is manufactured. In the gas sensor 100, the value of the target value V1* is the value determined in the above-mentioned target value determination process, thus reduction of the accuracy of measurement of a specific gas concentration due to a variation of the water concentration in the measurement-object gas is prevented.

The method for manufacturing a gas sensor in the second embodiment is also an example of the method for manufacturing the gas sensor 100 in the first embodiment described above, for example, the gas sensor 100 satisfying the above Expression (1) and Expression (2), or the gas sensor 100 satisfying the above Expression (3) and Expression (4). In the second embodiment, an example of the method for determining an adjustment voltage target value for control of a sensor element of the present invention will also be clarified by describing the method for manufacturing the sensor element 101 and the gas sensor 100.

In the method for determining an adjustment voltage target value of the sensor element 101 of the present embodiment described in detail above, the adjustment voltage target value is determined so that the smaller the diffusion resistance B relative to the diffusion resistance A, the adjustment voltage target value (the target value V1* in this case) tends to be lower, thus the adjustment voltage target value is easily determined to an optimal point or a value close to an optimal point. Controlling the sensor element 101 based on thus determined adjustment voltage target value facilitates prevention of reduction in the accuracy of measurement of a specific gas concentration due to a variation of the water concentration in the measurement-object gas.

Also, the limiting current values La, Lb as values correlated with the diffusion resistances A, B are measured, and the adjustment voltage target value is determined so that the greater the limiting current value Lb relative to the measured limiting current value La, the adjustment voltage target value tends to be lower. Thus, instead of measuring the diffusion resistance A and the diffusion resistance B, the limiting current value La and the limiting current value Lb are measured, and the adjustment voltage target value can be determined to an optimal point or a value close to an optimal point.

In addition, the adjustment voltage target value is determined so that the greater the value R (=Lb*Lb/La), the adjustment voltage target value tends to be lower. Since the value R has a linear relationship with the optimal point of the adjustment voltage target value, using the value R, the adjustment voltage target value is easily determined to an optimal point or a value close to an optimal point.

The adjustment voltage target value is then determined as the value corresponding to the measured limiting current values La, Lb based on the prepared corresponding relationship in advance between the limiting current values La, Lb and the adjustment voltage target value. Thus, an appropriate adjustment voltage target value can be easily determined using the prepared corresponding relationship in advance.

### Third Embodiment

Another embodiment of the method for manufacturing a gas sensor, including the method for determining an adjustment voltage target value for control of a sensor element of the present invention will be described. The method for manufacturing a gas sensor of the present embodiment is the same as the method for manufacturing a gas sensor of the second embodiment except that the target value determination process is different. Thus, a description of any process other than the target value determination process will be omitted.

The target value determination process of the present embodiment includes: a step (b1) of setting the target value V2* (an example of the measurement voltage target value) to a predetermined value, setting the target value V1* (an example of the adjustment voltage target value) to a temporary target value, and measuring a first measurement pump current and a second measurement pump current, the first measurement pump current being the pump current Ip2 which flows through the measurement pump cell 41 when the adjustment-pump control process and the measurement-pump control process are performed on a first gas with a water concentration of a first concentration Ch1 as the measurement-object gas, the second measurement pump current being the pump current Ip2 which flows through the measurement pump cell 41 when the adjustment-pump control process and the measurement-pump control process are performed on a second gas with a water concentration of a second concentration Ch2 lower than the first concentration Ch 1 as the measurement-object gas; and a step (b2) of changing the temporary target value and performing the step (b1) for multiple times, and determining a temporary target value to be the target value V1* for control of the sensor element 101, the temporary target value causing the discrepancy between the first measurement pump current and the second measurement pump current to fall within a predetermined minute range.

As described in the first embodiment, the gas sensor element 101 has an optimal point for the target value V1*, the optimal point being capable of preventing reduction in the accuracy of measurement of a specific gas concentration due to a variation of the water concentration in the measurement-object gas. Thus, when a temporary target value of the target value V1* is an optimal point or a value close to an optimal point, the first measurement pump current and the second measurement pump current measured in the step (b1) have the same value or values close to each other. Thus, an appropriate target value V1* can be found by changing the temporary target value and performing the step (b1) for multiple times. In the step (b2), in this manner, the temporary target value is changed and the step (b1) is performed for multiple times, and the temporary target value causing the discrepancy between the first measurement pump current and the second measurement pump current to fall within a predetermined minute range is determined to be the target value V1* for control of the sensor element 101, thus the target value V1* can be determined to an optimal point or a value close to an optimal point. Controlling the sensor element 101 based on thus determined target value V1* can prevent reduction in the accuracy of measurement of a specific gas concentration due to a variation of the water concentration in the measurement-object gas. When the target value V1* is determined in this manner, it is not necessary to measure the diffusion resistances A, B or the limiting current values La, Lb as in the second embodiment.

A predetermined minute range can be defined in advance so that the effect of the discrepancy between the first measurement pump current and the second measurement pump current on the accuracy of measurement of a specific gas concentration is within an acceptable range. As the value representing the discrepancy between the first measurement pump current and the second measurement pump current, the above-mentioned ΔIp2offset, that is, the difference between the first measurement pump current and the second measurement pump current may be used. For example, in the step (b2), it is determined whether the absolute value of the ΔIp2offset is less than or equal to a predetermined threshold value, (for example, whether the absolute value is less than or equal to 3 ppm, or whether the absolute value is less than or equal to 1 ppm in terms of NO concentration conversion), and when an affirmative determination is made, the temporary target value may be determined to be the target value V1* for control of the sensor element 101. As the value representing the discrepancy between the first measurement pump current and the second measurement pump current, the ratio of the first measurement pump current to the second measurement pump current may be used. For example, in step (b2), it is determined whether the ratio is within a predetermined range which is close to value 1, and when an affirmative determination is made, the temporary target value may be determined to the target value V1* for control of the sensor element 101.

The first gas and the second gas used in the step (b1) may have the same composition as that of the first gas and the second gas (the measurement-object gas at the time of calculation of the ΔIp2offset illustrated in FIG. 4) described in the first embodiment. However, without being limited to this, it is sufficient that the water concentration (second concentration Ch2) of the second gas be lower than the water concentration (first concentration Ch1) of the first gas. For example, the first gas and the second gas may differ in at least one of oxygen concentration and NO concentration. However, the first gas and the second gas preferably have the same composition as much as possible except for the water concentration. Thus, the first gas and the second gas preferably have the same oxygen concentration and the same NO concentration. Each of the first gas and the second gas preferably has an NO concentration of 0 ppm. In the step (b1), either one of the first measurement pump current and the second measurement pump current may be measured first.

The predetermined value of the target value V2* at the time of measurement of the first measurement pump current and the second measurement pump current in the step (b1) is preferably the same as or close to the target value V2* for the measurement-pump control process when the sensor element 101 is actually used for detection of a specific gas concentration. In the present embodiment, the predetermined value of the target value V2* is 400 mV which is the same as at the time of use of the sensor element 101. The predetermined value may be e.g., 380 mV or higher and 420 mV or lower.

As a result, the gas sensor 100 of the above-described first embodiment, for example, the gas sensor 100 satisfying the Expression (1) and Expression (2), and the gas sensor 100 satisfying the Expression (3) and Expression (4) can be manufactured by the manufacturing method including the target value determination process of the third embodiment.

In the method for determining an adjustment voltage target value of the sensor element 101 of the present embodiment described in detail above, the temporary target value of the adjustment voltage target value (the target value V1* in this case) is changed and the step (b1) is performed for multiple times, and the temporary target value causing the discrepancy between the first measurement pump current and the second measurement pump current to fall within a predetermined minute range is determined to be the adjustment voltage target value for control of the sensor element 101, thus the adjustment voltage target value can be determined to an optimal point or a value close to an optimal point. Controlling the sensor element 101 based on thus determined adjustment voltage target value can prevent reduction in the accuracy of measurement of a specific gas concentration due to a variation of the water concentration in the measurement-object gas.

Note that the present invention is not limited to the above-described embodiment at all, and it is needless to say that the present invention can be carried out in various forms as long as the forms belong to the technical scope of the present invention.

For example, in the above-described first to third embodiments, the oxygen concentration adjustment chamber includes the first internal cavity 20 and the second internal cavity 40; however, without being limited to this, for example, the oxygen concentration adjustment chamber may further include another internal cavity, or one of the first internal cavity 20 and the second internal cavity 40 may be omitted. Similarly, in the above-described first to third embodiments, the adjustment pump cell includes the main pump cell 21 and the auxiliary pump cell 50; however, without being limited to this, for example, the adjustment pump cell may further include another pump cell, or one of the main pump cell 21 and the auxiliary pump cell 50 may be omitted. For example, when the oxygen concentration of the measurement-object gas can be sufficiently reduced only by the main pump cell 21, the auxiliary pump cell 50 may be omitted. When the auxiliary pump cell 50 is omitted, the controller 96 only need to perform the main-pump control process as the adjustment-pump control process. In the main-pump control process, the above-described setting of the target value V0* based on the pump current Ip1 may be omitted. Specifically, a predetermined target value V0* is pre-stored in the storage unit 98, and the controller 96 may control the main pump cell 21 by feed-back controlling the voltage Vp0 of the variable power source 24 so that the voltage V0 reaches the target value V0*. When the auxiliary pump cell 50 is omitted, the target value V0* corresponds to the adjustment voltage target value (the target value V1* in the first to third embodiments). Thus, for example, V1* in Expression (1) to Expression (5) indicates the target value V0*. When the oxygen concentration adjustment chamber further includes another internal cavity in addition to the first internal cavity 20 and the second internal cavity 40, and the adjustment pump cell includes three or more pump cells, the target value of the voltage in the control process of the most downstream pump cell among the three or more pump cells, in other words, the pump cell disposed closest to the measurement chamber corresponds to the adjustment voltage target value (the target value V1* in the first to third embodiments). When the adjustment pump cell includes three or more pump cells, the limiting current value La to be measured in the second embodiment may be the limiting current value of any pump cell among the three or more pump cells.

In the above-described first to third embodiments, the sensor element 101 of the gas sensor 100 includes the first internal cavity 20, the second internal cavity 40, and the third internal cavity 61, but is not limited thereto. For example, as the sensor element 201 of FIG. 6, the third internal cavity 61 may not be included. In the sensor element 201 of the modification illustrated in FIG. 6, the gas inlet 10, the first diffusion control section 11, the buffer space 12, the second diffusion control section 13, the first internal cavity 20, the third diffusion control section 30, and the second internal cavity 40 are formed adjacently in that order in a communicating manner between the lower surface of the second solid electrolyte layer 6 and the upper surface of the first solid electrolyte layer 4. The measurement electrode 44 is disposed on the upper surface of the first solid electrolyte layer 4 in the second internal cavity 40. The measurement electrode 44 is covered by the fourth diffusion control section 45. The fourth diffusion control section 45 is a film made of a ceramic porous body such as alumina (Al₂O₃). As with the fourth diffusion control section 60 of the above-described embodiment, the fourth diffusion control section 45 has a function of limiting the amount of NOx which flows into the measurement electrode 44. The fourth diffusion control section 45 also functions as a protective film of the measurement electrode 44. The ceiling electrode portion 51a of the auxiliary pump electrode 51 is formed to a position immediately above the measurement electrode 44. Even with thus configured sensor element 201, as in the above-described embodiment, NOx concentration can be detected, for example, based on the pump current Ip2. In this case, the vicinity of the measurement electrode 44 functions as a measurement chamber. Also, the composite resistance of the first diffusion control section 11, the second diffusion control section 13, the third diffusion control section 30 and the fourth diffusion control section 45 corresponds to the diffusion resistance B.

In the above-described second embodiment, in the step (a2) of the target value determination process, as a prepared corresponding relationship in advance between the limiting current values La, Lb and the target value V1*, a corresponding relationship between the value R and the target value V1* has been exemplified, but is not limited thereto. For example, a corresponding relationship between the ratio (=Lb/La) of the limiting current value Lb to the limiting current value La, and the target value V1* may be prepared in advance. Alternatively, without using a value (such as the value R or the above-mentioned ratio) calculated from the limiting current value La and the limiting current value Lb, a corresponding relationship between the limiting current value La and the limiting current value Lb, and the target value V1* may be prepared in advance.

In the above-described second embodiment, the limiting current value La measured in the step (a1) is the limiting current value of the pump current lp0 which flows through the main pump cell 21 when the main pump cell 21 pumps out oxygen from the first internal cavity 20, but is not limited thereto. The limiting current value La may be the limiting current value of the adjustment pump current which flows through the adjustment pump cell when the adjustment pump cell pumps out oxygen from the oxygen concentration adjustment chamber. For example, in the step (a1), as the limiting current value La, the limiting current value of the pump current Ip1 may be measured, which flows through the auxiliary pump cell 50 when the auxiliary pump cell 50 pumps out oxygen from the second internal cavity 40. The limiting current value of the pump current Ip1 is correlated with the diffusion resistance from the outside of the sensor element 101 to the second internal cavity 40 (an example of the oxygen concentration adjustment chamber). Thus, as with the limiting current value of the pump current Ip0, the limiting current value of the pump current Ip1 can be utilized as a value related to the degree of upstream movement (diffusion) of hydrogen relative to the measurement-object gas flow portion, the hydrogen being produced in the vicinity of the auxiliary pump electrode 51. In this case, the composite resistance of the first diffusion control section 11, the second diffusion control section 13 and the third diffusion control section 30 which are located upstream of the second internal cavity 40 of the measurement-object gas flow portion corresponds to the diffusion resistance A.

The target value determination process (the method for determining an adjustment voltage target value) of the second, third embodiments described above may be performed at a timing other than the time of manufacturing of a sensor element and the time of manufacturing of a gas sensor. For example, for the gas sensor 100 with an adjustment voltage target value already determined, mounted on a vehicle, the target value determination process of the second embodiment may be performed to calibrate the adjustment voltage target value. When the measurement-object gas has an oxygen concentration which allows the limiting current values La, Lb to be measured, an adjustment voltage target value can be determined by performing the steps (a1), (a2) described above. For example, the target value determination process may be performed at a target value determination timing at which the measurement-object gas is assumed to be an atmospheric atmosphere. For example, the target value determination timing may be a timing at which the measurement-object gas is assumed to be an exhaust gas at the time of fuel cut of an internal combustion engine. As described above, the controller 96 can detect an oxygen concentration in the measurement-object gas based on the pump current lp0 which is caused to flow due to the main-pump control process, thus can detect a target value determination timing based on the pump current lp0. For example, when the pump current lp0 suddenly increases or increases to a value exceeding a predetermined threshold value, the controller 96 may determine that a target value determination timing has reached. At the target value determination timing, the controller 96 may determine an adjustment voltage target value in the following manner. First, the controller 96 stops the adjustment-pump control process and the measurement-pump control process. Next, the controller 96 performs the step (a1) to measure the limiting current value La and the limiting current value Lb. As in the first embodiment, the controller 96 may measure the limiting current value La by gradually increasing the voltage Vp0. Alternatively, the controller 96 may set the target value V0* to a relatively high predetermined value (a value higher than the target value V0* set in the main-pump control process at the time of measurement of NOx concentration) which causes the pump current lp0 to be a limiting current, and perform the same process as the main-pump control process to measure the pump current lp0 which flows at this point as the limiting current value La. Similarly, as in the first embodiment, the controller 96 may measure the limiting current value Lb by gradually increasing the voltage Vp2, or may set the target value V2* to a relatively high predetermined value which causes the pump current Ip2 to be a limiting current, and perform the measurement-pump control process to measure the pump current Ip2 which flows at this point as the limiting current value Lb. As described above, the limiting current value of the pump current Ip1 may be measured as the limiting current value La. The controller 96 then performs the step (a2) to determine the target value V1* based on the limiting current values La, Lb measured in the step (a1). In the step (a2), a corresponding relationship between limiting current values La, Lb, and an appropriate target value V1* is prepared in advance and stored in the storage unit 98, the limiting current values La, Lb being measured in a state where the measurement-object gas is an exhaust gas (e.g., an atmosphere) at the time of fuel cut, then the target value V1* is preferably determined based on the stored corresponding relationship and the limiting current values La, Lb measured in the step (a1). The controller 96 calibrates the target value V1* by overwriting the currently set value of the target value V1* with thus determined target value V1*. In the sensor element 101, the diffusion resistance of the first diffusion control section 11, the second diffusion control section 13, the third diffusion control section 30, and the fourth diffusion control section 60 may vary due to use, and thus the balance between the diffusion resistances A, B may be changed. Even in this situation, an appropriate target value V1* is likely to be maintained by the controller 96 calibrating the target value V1*, thus it is possible to prevent increase of the effect of reduction in the accuracy of measurement of a specific gas concentration due to a variation of the water concentration in the measurement-object gas with use of the sensor element 101.

In the above-described embodiment, the outer pump electrode 23 plays a role as the electrode (also referred to as an outer main pump electrode) to be paired with the inner pump electrode 22 in the main pump cell 21, plays a role as the electrode (also referred to as an outer auxiliary pump electrode) to be paired with the auxiliary pump electrode 51 in the auxiliary pump cell 50, and plays a role as the electrode (also referred to as an outer measurement electrode) to be paired with the measurement electrode 44 in the measurement pump cell 41; however, the configuration is not limited thereto. One or more of the outer main pump electrode, the outer auxiliary pump electrode, and the outer measurement electrode may be provided separately from the outer pump electrode 23 outside the element body so as to be in contact with the measurement-object gas.

In the above-described embodiment, the sensor element 101 detects the NOx concentration in the measurement-object gas; however, as long as the sensor element 101 detects the concentration of a specific gas in the measurement-object gas, the configuration is not limited thereto. For example, not only NOx, but also another oxide concentration may serve as the specific gas concentration. When the specific gas is an oxide, as in the above-described embodiment, oxygen is produced when the specific gas itself is reduced in the third internal cavity 61, thus the measurement pump cell 41 can detect the specific gas concentration by obtaining a detection value (e.g., the pump current Ip2) corresponding to the oxygen. Alternatively, the specific gas may be a non-oxide such as ammonia. When the specific gas is a non-oxide, the specific gas is converted to an oxide (for example, ammonia is converted to NO), thereby producing oxygen when the gas after the conversion is reduced in the third internal cavity 61, thus the measurement pump cell 41 can detect the specific gas concentration by obtaining a detection value (e.g., the pump current Ip2) corresponding to the oxygen. For example, the inner pump electrode 22 in the first internal cavity 20 functions as a catalyst, thus ammonia can be converted to NO in the first internal cavity 20. The specific gas may be a predetermined gas other than oxygen. The specific gas may be a predetermined gas other than oxygen and carbon dioxide. The specific gas may be one of NOx and ammonia.

In the above-described embodiment, the element body of the sensor element 101 is a layered body having a plurality of solid electrolyte layers (layers 1 to 6), but is not limited thereto. The element body of the sensor element 101 may include at least one oxygen-ion-conductive solid electrolyte layer. For example, in FIG. 1, the layers 1 to 5 other than the second solid electrolyte layer 6 may be layers (e.g., layers composed of alumina) composed of a material other than that of solid electrolyte layers. In this case, the electrodes of the sensor element 101 may be disposed in the second solid electrolyte layer 6. For example, the measurement electrode 44 in FIG. 1 may be disposed on the lower surface of the second solid electrolyte layer 6. Also, the reference gas inlet space 43 may be provided in the spacer layer 5 instead of the first solid electrolyte layer 4, the reference gas inlet layer 48 may be provided between the second solid electrolyte layer 6 and the spacer layer 5 instead of between the first solid electrolyte layer 4 and the third substrate layer 3, and the reference electrode 42 may be provided rearward of the third internal cavity 61 and on the lower surface of the second solid electrolyte layer 6.

In the above-described embodiment, the controller 96 sets (feedback-controls) the target value V0* of the voltage V0 based on the pump current Ip1 so that the pump current Ip1 reaches the target value Ip1*, and feedback-controls the voltage Vp0 so that the voltage V0 reaches the target value V0*, but may perform another control. For example, the controller 96 may feedback-control the voltage Vp0 based on the pump current Ip1 so that the pump current Ip1 reaches the target value Ip1*. In other words, the controller 96 may omit acquisition of the voltage V0 from the main pump control oxygen partial pressure detection sensor cell 80 and setting of the target value V0*, and may directly control the voltage Vp0 (eventually, control the pump current Ip0) based on the pump current Ip1.

In the above-described embodiments, the sensor element 101 may include a porous protective layer that covers at least part (e.g., the distal end of the element body) of the element body. The porous protective layer may contain, as constituent particles, ceramic particles of at least one of alumina, zirconia, spinel, cordierite, titania and magnesia. The porous protective layer may cover the gas inlet 10 and the outer pump electrode 23. When the porous protective layer covers the gas inlet 10, the aforementioned diffusion resistances A, B also include the diffusion resistance of the porous protective layer. Also, in this case, the aforementioned limiting current values La, Lb have values correlated with the diffusion resistances A, B.

What is claimed is:

1. A gas sensor comprising a sensor element, and a control device,
the sensor element including:
an element body that includes an oxygen-ion-conductive solid electrolyte layer, and is internally provided with a measurement-object gas flow portion that introduces a measurement-object gas and causes the measurement-object gas to flow therethrough;
a measurement pump cell including an inner measurement electrode disposed in a measurement chamber of the measurement-object gas flow portion, the measurement pump cell being configured to pump oxygen from the measurement chamber to an outside of the element body;
an adjustment pump cell including an inner adjustment electrode disposed in an oxygen concentration adjustment chamber located upstream of the measurement chamber in the measurement-object gas flow portion, the adjustment pump cell being configured to adjust an oxygen concentration in the oxygen concentration adjustment chamber; and
a reference electrode disposed inside the element body so as to be in contact with a reference gas which serves as a reference for detection of a specific gas concentration that is a concentration of a specific gas in the measurement-object gas,
wherein the control device is configured to perform an adjustment-pump control process and a measurement-pump control process, the adjustment-pump control process for adjusting an oxygen concentration in the oxygen concentration adjustment chamber by controlling the adjustment pump cell so that an adjustment voltage that is a voltage across the reference electrode and the inner adjustment electrode reaches an adjustment voltage target value, the measurement-pump control process for pumping out oxygen from the measurement chamber by controlling the measurement pump cell so that a measurement voltage that is a voltage across the reference electrode and the inner measurement electrode reaches a measurement voltage target value, and
let V1* [mV] be the adjustment voltage target value, then following Expression (1) and Expression (2) are satisfied:
$$V 1 ⋆ \leq - 25.7 * R + 436. \qquad ( 1 )$$
$$V 1 ⋆ \geq - 35.1 * R + 406.6 \qquad ( 2 )$$
where R=Lb*Lb/La,
La is a limiting current value [mA] of an adjustment pump current which flows through the adjustment pump cell when the adjustment pump cell pumps out oxygen from the oxygen concentration adjustment chamber with the sensor element exposed to the measurement-object gas which is a nitrogen-based gas with an oxygen concentration of 20.5%, a water concentration of 3%, and an NO concentration of 0 ppm, and
Lb is a limiting current value [μA] of a measurement pump current which flows through the measurement pump cell when the measurement pump cell pumps out oxygen from the measurement chamber with the sensor element exposed to the measurement-object gas which is a nitrogen-based gas with an oxygen concentration of 0%, a water concentration of 3%, and an NO concentration of 500 ppm.

2. The gas sensor according to claim 1,
wherein following Expression (3) and Expression (4) are satisfied:
$$V 1 ⋆ \leq - 18.9 * R + 419.2 \qquad ( 3 )$$
$$V 1 ⋆ \geq - 16.2 * R + 403.4 . \qquad ( 4 )$$

3. The gas sensor according to claim 1,
wherein the oxygen concentration adjustment chamber includes a first internal cavity, and a second internal cavity provided downstream of the first internal cavity and upstream of the measurement chamber,
the adjustment pump cell includes a main pump cell configured to adjust an oxygen concentration in the first internal cavity, and an auxiliary pump cell configured to adjust an oxygen concentration in the second internal cavity,
the inner adjustment electrode includes an inner main pump electrode that is disposed in the first internal cavity and constitutes part of the main pump cell, and an inner auxiliary pump electrode that is disposed in the second internal cavity and constitutes part of the auxiliary pump cell,
the adjustment voltage is a voltage across the reference electrode and the inner auxiliary pump electrode, and
the limiting current value La is a limiting current value of a main pump current which flows through the main pump cell when the main pump cell pumps out oxygen from the first internal cavity with the sensor element exposed to the measurement-object gas which is a nitrogen-based gas with an oxygen concentration of 20.5%, a water concentration of 3%, and an NO concentration of 0 ppm.

4. A method for determining an adjustment voltage target value for control of a sensor element,
the sensor element including:
an element body that includes an oxygen-ion-conductive solid electrolyte layer, and is internally provided with a measurement-object gas flow portion that introduces a measurement-object gas and causes the measurement-object gas to flow therethrough;
a measurement pump cell including an inner measurement electrode disposed in a measurement chamber of the measurement-object gas flow portion, the measurement pump cell being configured to pump oxygen from the measurement chamber to an outside of the element body;
an adjustment pump cell including an inner adjustment electrode disposed in an oxygen concentration adjustment chamber located upstream of the measurement chamber in the measurement-object gas flow portion, the adjustment pump cell being configured to adjust an oxygen concentration in the oxygen concentration adjustment chamber; and
a reference electrode disposed inside the element body so as to be in contact with a reference gas which serves as a reference for detection of a specific gas concentration that is a concentration of a specific gas in the measurement-object gas,
wherein the adjustment voltage target value is a target value of an adjustment voltage when a specific gas concentration that is a concentration of a specific gas in the measurement-object gas is detected using the sensor element, the adjustment voltage being a voltage across the reference electrode and the inner adjustment electrode at a time of feedback control of the adjustment pump cell to adjust the oxygen concentration in the oxygen concentration adjustment chamber,
the method including
a step (a) of determining the adjustment voltage target value so that lower a diffusion resistance B relative to a diffusion resistance A, the adjustment voltage target value tends to be lower, where the diffusion resistance A is a diffusion resistance from an outside of the sensor element to the oxygen concentration adjustment chamber, and the diffusion resistance B is a diffusion resistance from the outside of the sensor element to the measurement chamber.

5. The method for determining an adjustment voltage target value for control of a sensor element according to claim 4,
wherein the step (a) includes:
a step (a1) of measuring, as a value correlated with the diffusion resistance A, a limiting current value La of an adjustment pump current which flows through the adjustment pump cell when the adjustment pump cell pumps out oxygen from the oxygen concentration adjustment chamber, and as a value correlated with the diffusion resistance B, a limiting current value Lb of a measurement pump current which flows through the measurement pump cell when the measurement pump cell pumps out oxygen from the measurement chamber; and
a step (a2) of determining the adjustment voltage target value so that for the limiting current values La, Lb measured in the step (a1), greater the limiting current value Lb relative to the limiting current value La, the adjustment voltage target value tends to be lower.

6. The method for determining an adjustment voltage target value for control of a sensor element according to claim 5,
wherein in the step (a2), the adjustment voltage target value is determined so that the adjustment voltage target value tends to be lower for a greater value R expressed below:
$$R = Lb ⋆ Lb / La .$$

7. The method for determining an adjustment voltage target value for control of a sensor element according to claim 5,
wherein in the step (a2), the adjustment voltage target value is determined as a value corresponding to the limiting current values La, Lb measured in the step (a1) based on a prepared corresponding relationship in advance between the limiting current values La, Lb and the adjustment voltage target value.

8. The method for determining an adjustment voltage target value for control of a sensor element according to claim 5,
wherein the oxygen concentration adjustment chamber includes a first internal cavity, and a second internal cavity provided downstream of the first internal cavity and upstream of the measurement chamber,
the adjustment pump cell includes a main pump cell configured to adjust an oxygen concentration in the first internal cavity, and an auxiliary pump cell configured to adjust an oxygen concentration in the second internal cavity,
the inner adjustment electrode includes an inner main pump electrode that is disposed in the first internal cavity and constitutes part of the main pump cell, and an inner auxiliary pump electrode that is disposed in the second internal cavity and constitutes part of the auxiliary pump cell,
the adjustment voltage is a voltage across the reference electrode and the inner auxiliary pump electrode, and
the limiting current value La is a limiting current value of a main pump current which flows through the main pump cell when the main pump cell pumps out oxygen from the first internal cavity.

9. A method for determining an adjustment voltage target value for control of a sensor element,
the sensor element including:
an element body that includes an oxygen-ion-conductive solid electrolyte layer, and is internally provided with a measurement-object gas flow portion that introduces a measurement-object gas and causes the measurement-object gas to flow therethrough;
a measurement pump cell including an inner measurement electrode disposed in a measurement chamber of the measurement-object gas flow portion, the measurement pump cell being configured to pump oxygen from the measurement chamber to an outside of the element body;
an adjustment pump cell including an inner adjustment electrode disposed in an oxygen concentration adjustment chamber located upstream of the measurement chamber in the measurement-object gas flow portion, the adjustment pump cell being configured to adjust an oxygen concentration in the oxygen concentration adjustment chamber; and
a reference electrode disposed inside the element body so as to be in contact with a reference gas which serves as a reference for detection of a specific gas concentration that is a concentration of a specific gas in the measurement-object gas,
wherein the sensor element is configured to, when a specific gas concentration that is a concentration of a specific gas in the measurement-object gas is detected, perform an adjustment-pump control process and a measurement-pump control process, the adjustment-pump control process for adjusting the oxygen concentration in the oxygen concentration adjustment chamber by controlling the adjustment pump cell so that an adjustment voltage that is a voltage across the reference electrode and the inner adjustment electrode reaches the adjustment voltage target value, the measurement-pump control process for pumping out oxygen from the measurement chamber by controlling the measurement pump cell so that a measurement voltage that is a voltage across the reference electrode and the inner measurement electrode reaches a measurement voltage target value,
the method including:
a step (b1) of setting the measurement voltage target value to a predetermined value, setting the adjustment voltage target value to a temporary target value, and measuring a first measurement pump current and a second measurement pump current, the first measurement pump current being caused to flow through the measurement pump cell when the adjustment-pump control process and the measurement-pump control process are performed on a first gas as the measurement-object gas, the first gas having a water concentration of a first concentration Ch1, the second measurement pump current being caused to flow through the measurement pump cell when the adjustment-pump control process and the measurement-pump control process are performed on a second gas as the measurement-object gas, the second gas having a water concentration of a second concentration Ch2 lower than the first concentration Ch 1; and a step (b2) of changing the temporary target value and performing the step (b1) for multiple times, and determining the temporary target value to be the adjustment voltage target value for control of the sensor element, the temporary target value causing a discrepancy between the first measurement pump current and the second measurement pump current to fall within a predetermined minute range.

10. A method for manufacturing a sensor element, including the method for determining an adjustment voltage target value for control of a sensor element according to claim 4, and being associated with the adjustment voltage target value,
the method comprising:
a manufacturing process for manufacturing the sensor element;
a target value determination process for determining the adjustment voltage target value by performing, on the manufactured sensor element, the steps of the method for determining the adjustment voltage target value for control of the sensor element; and
an association process for associating the manufactured sensor element with the determined adjustment voltage target value.

11. A method for manufacturing a gas sensor, comprising:
the processes of the method for manufacturing a sensor element according to claim 10; and
a connection process for connecting the sensor element associated with the adjustment voltage target value to a control device that feedback-controls the adjustment pump cell so that a voltage across the reference electrode and the inner adjustment electrode reaches the associated adjustment voltage target value.

12. A method for manufacturing a sensor element, including the method for determining an adjustment voltage target value for control of a sensor element according to claim 9, and being associated with the adjustment voltage target value,
the method comprising:
a manufacturing process for manufacturing the sensor element;
a target value determination process for determining the adjustment voltage target value by performing, on the manufactured sensor element, the steps of the method for determining the adjustment voltage target value for control of the sensor element; and
an association process for associating the manufactured sensor element with the determined adjustment voltage target value.

13. A method for manufacturing a gas sensor, comprising:
the processes of the method for manufacturing a sensor element according to claim 12; and
a connection process for connecting the sensor element associated with the adjustment voltage target value to a control device that feedback-controls the adjustment pump cell so that a voltage across the reference electrode and the inner adjustment electrode reaches the associated adjustment voltage target value.
